(19) United States
(12) Patent Application Publication
(10) Pub. No.: US 2022/0298491 A1
(43) Pub. Date: Sep. 22, 2022

(54) PLURIPOTENT STEM CELL DERIVED DENDRITIC CELLS AND ENGINEERED DENDRITIC CELLS FOR CANCER IMMUNOTHERAPY

(71) Applicant: Therapeutic Solutions International, Inc., Oceanside, CA (US)

(72) Inventors: Thomas E. ICHIM, Oceanside, CA (US); Famela RAMOS, Oceanside, CA (US); James VELTMEYER, Oceanside, CA (US); Timothy G. DIXON, Oceanside, CA (US)

(73) Assignee: Therapeutic Solutions International, Inc., Oceanside, CA (US)

(21) Appl. No.: 17/696,763

(22) Filed: Mar. 16, 2022

Related U.S. Application Data
(60) Provisional application No. 63/161,526, filed on Mar. 16, 2021.

(51) Int. Cl. C12N 5/074; A61K 39/00

(52) U.S. Cl. CPC C12N 5/0696; A61K 39/0011; C12N 2510/00; C12N 2501/602; C12N 2501/603; C12N 2501/604; C12N 2501/606; C12N 2501/605; C12N 2501/608

(57) ABSTRACT

Disclosed are populations of dendritic cells generated from stem cells capable of inducing immunity towards cancer. In one embodiment said dendritic cells are generated from allogeneic inducible pluripotent stem cells, for some uses, said pluripotent stem cells are genetically engineered/edited to induce cancer specific immunity and/or resist immunosuppressive effect of tumor derived microenvironment. In one embodiment pluripotent stem cells are transfected with cancer stem cell antigens such as BORIS and/or NR2F6.

### CROSS-REFERENCE TO RELATED APPLICATIONS

This application claims priority to U.S. Provisional Application Ser. No. 63/161,526, filed on Mar. 16, 2021, entitled “Pluripotent Stem Cell Derived Dendritic Cells and Engineered Dendritic Cells for Cancer Immunotherapy”, which is incorporated herein by reference in its entirety.

### FIELD OF THE INVENTION

The invention belongs to the area of immune modulation, more specifically the invention belongs to the field of utilizing dendritic cells generated from stem cells for stimulating a subjects immune system. These methods are useful for treating cancer.

### BACKGROUND OF THE INVENTION

Dendritic cells (DC) were originally identified by Ralph Steinman as bone marrow derived professional antigen presenting cells, being the only cell of the immune system capable of activating naïve T cells [1]. Subsequent studies have shown that DC act as a critical bridge between the innate immune system, which is constantly patrolling for various “danger” signals such as toll like receptor (TLR) agonists that are associated with tissue injury or pathogenic threat. In contrast to other antigen presenting cells such as the macrophage or the B cell, DC exhibit magnitudes of higher ability to stimulate T cell responses both in antigen specific systems, as well as in polyclonal experiments such as in mixed lymphocyte reaction [2]. It is known that in peripheral tissues (outside of lymph nodes), DCs capture antigens through several complementary mechanisms including phagocytosis and receptor mediated endocytosis. Immature DC are known to possess high degree of phagocytic activity and low levels of antigen presenting activity. Normally, DCs in peripheral tissues are immature. These immature DCs have the ability to efficiently capture antigens; they can accumulate MHC class II molecules in the late endosome-lysosomal compartment; they can express low levels of co-stimulatory molecules; they can express a unique set of chemokine receptors (such as CCR7) that allow their migration to lymphoid tissues; and they have a limited capacity for secreting cytokines [3].

Once DC are activated, by a stimulatory signal such as a toll like receptor agonist, phagocytic activity decreases and the DC then migrate into the draining lymph nodes through the afferent lymphatics. During the trafficking process, DC degrade ingested proteins into peptides that bind to both MHC class I molecules and MHC class II molecules. This allows the DC to: a) perform cross presentation in that they ingest exogenous antigens but present peptides in the MHC I pathway; and b) activate both CD8 (via MHC I) and CD4 (via MHC II). Interestingly, lipid antigens are processed via different pathways and are loaded onto non-classical MHC molecules of the CD1 family [4].

DCs promptly respond to environmental signals and differentiate into mature DCs that can efficiently launch immune responses. As stated above, maturation is associated with the downregulation of antigen-capture activity, the increased expression of surface MHC class II molecules and costimulatory molecules, the ability to secrete cytokines as well as the acquisition of CCR7, which allows migration of the DC into the draining lymph node. The ligation of the costimulatory receptor CD40 (also known as TNFRSF5) is an essential signal for the differentiation of immature DCs into fully mature DCs that are able to launch adaptive T cell-mediated immunity [5]. However, DC maturation alone does not result in a unique DC phenotype. Instead, the different signals that are provided by different microbes or viruses either directly or through the surrounding immune cells induce DCs to acquire distinct phenotypes that eventually contribute to different immune responses. Indeed, DC maturation varies according to different microbes because microbes express different pathogen associated molecular patterns (PAMPs) that trigger distinct DC molecular sensors, which are called pattern recognition receptors (PPRs). Strikingly, although most microbes activate DCs, a few can block DC maturation through various pathways [6]. Tissue-localized DCs can also be polarized into distinct phenotypes by the products released from surrounding immune cells that respond to injury. For example, γd-T cells and NKcells release interferon-γ (IFNγ), mast cells release pre-formed IL-4 and TNF, pDCs secrete IFNa, stromal cells secrete IL-15 and thymic stromal lymphopoietin (TSLP), and so on. These cytokines induce the differentiation of progenitor cells or of precursor cells such as monocytes into distinct inflammatory DCs that yield unique types of T cells. On interaction of CD4 and CD8 T cells with DC, these cells can subsequently differentiate into antigen-specific effector T cells with different functions. CD4 T cells can become T helper 1 (TH1) cells, TH2 cells, TH17 cells or T follicular helper (T) cells that help B cells to differentiate into antibody-secreting cells, as well as Treg cells. Naive C D8 T cells can give rise to effector cytotoxic T lymphocytes (CTLs).

An interesting activity of DC is that in addition to stimulating immune responses through the activation of naïve T cells, DC are also able to act as inhibitory cells. This is either directly, through inhibition of T cell activation and/or induction of T cell anergy [7], as well as indirectly through stimulation of T regulatory (Treg) cells [8, 9]. It is interesting that not only Treg cells, but also anergic T cells are capable of inhibiting DC activation [10-12], thus possibly stimulating a self maintaining immune regulatory feedback loop. In fact, such a scenario has been previously reported where Treg stimulate immature DC and the immature DC in turn stimulate production of new Treg cells [13, 14].

### SUMMARY

Preferred methods involve generating a dendritic cell from a stem cell possessing enhanced ability to induce anticancer immunity, wherein said dendritic cell is obtained by the process of: a) selecting a stem cell population; b) genetically modifying said stem cell population to endow enhanced anticancer activity towards said stem cell; c) differentiating said stem cell into a dendritic cell population.

Preferred methods include embodiments wherein said stem cell is a pluripotent stem cell.

Preferred methods include embodiments wherein said pluripotent stem cell is an inducible pluripotent stem cell.

Preferred methods include embodiments wherein said inducible pluripotent stem cell is generated by culturing of a somatic cell in a placenta-derived cell-conditioned medium, wherein said medium is capable of inducing dedifferentiation of said somatic cell into induced pluripotent stem cells (iPSC).

Preferred methods include embodiments wherein the placenta-derived cell is a placenta-derived fibroblast-like cell, which is isolated from the human chorionic plate and cultured.

Preferred methods include embodiments wherein said placental derived cell is a placental derived monocyte.

Preferred methods include embodiments wherein said monocyte expresses Nanog.

Preferred methods include embodiments wherein said monocyte expresses IL-3 receptor.

Preferred methods include embodiments wherein said monocyte expresses CD90.

Preferred methods include embodiments wherein said monocyte expresses CD105.

Preferred methods include embodiments wherein said monocyte expresses KLF4.

Preferred methods include embodiments wherein said monocyte expresses c-met.

Preferred methods include embodiments wherein said monocyte expresses CD14.

Preferred methods include embodiments wherein said monocyte expresses CD64.

Preferred methods include embodiments wherein said monocyte expresses CD62L.

Preferred methods include embodiments wherein said monocyte expresses TNFR1.

Preferred methods include embodiments wherein said monocyte expresses TNFR2.

Preferred methods include embodiments wherein said monocyte expresses CD191.

Preferred methods include embodiments wherein said monocyte expresses CD191.

Preferred methods include embodiments wherein said monocyte expresses CX3CR1.

Preferred methods include embodiments wherein said monocyte expresses CCR5.

Preferred methods include embodiments wherein said monocyte expresses CCR7.

Preferred methods include embodiments wherein said monocyte expresses CCR8.

Preferred methods include embodiments wherein said monocyte expresses CXCR2.

Preferred methods include embodiments wherein said monocyte expresses CXCR4.

Preferred methods include embodiments wherein the somatic cell is transformed with a nucleic acid sequence encoding at least one protein selected from the group consisting of OCT4, SOX2, c-Myc and KLF4.

Preferred methods include embodiments wherein the somatic cell is at least one selected from the group consisting of fibroblasts, endothelial cells, epithelial cells and placental cells.

Preferred methods include embodiments wherein said somatic cells are selected from group of cells comprising of: dermal cells, endodermal cells, mesodermal cells, reprogrammed immune cells, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue reprogrammed immune cells, corneal keratocytes, tendon reprogrammed immune cells, bone marrow reticular tissue reprogrammed immune cells, nonepithelial reprogrammed immune cells, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells

Preferred methods include embodiments wherein preparing a placenta-derived cell-conditioned medium for inducing dedifferentiation is accomplished using these following steps: a placenta-derived cell culturing step of culturing human placenta-derived cells in a cell growth medium supplemented with a culture solution; and a culture solution collecting step of collecting the culture solution comprising the human placenta-derived cell culture from the cell growth medium.

Preferred methods include embodiments wherein the placenta-derived cell is a placenta-derived fibroblast-like cell, which is isolated from the human chorionic plate and cultured.

Preferred methods include embodiments wherein said placenta derived fibroblast-like cell expresses CD90.

Preferred methods include embodiments wherein said placenta derived fibroblast-like cell expresses CD73.

Preferred methods include embodiments wherein said placenta derived fibroblast-like cell expresses CD105.

Preferred methods include embodiments wherein said placenta derived fibroblast-like cell expresses VEGF-receptor 1.

Preferred methods include embodiments wherein said placenta derived fibroblast-like cell expresses VEGF-receptor 2.

Preferred methods include embodiments wherein said placenta derived fibroblast-like cell expresses TGF-beta receptor.

Preferred methods include embodiments wherein said placenta derived fibroblast-like cell expresses EGF receptor.

Preferred methods include embodiments wherein said placenta derived fibroblast-like cell expresses IGF-receptor.

Preferred methods include embodiments wherein said placenta derived fibroblast-like cell expresses HGF-receptor.

Preferred methods include embodiments wherein said placenta derived fibroblast-like cell expresses FGF-1-receptor.

Preferred methods include embodiments wherein said placenta derived fibroblast-like cell expresses FGF-2-receptor.

Preferred methods include embodiments wherein said placenta derived fibroblast-like cell expresses FGF-5-receptor.

Preferred methods include embodiments wherein said placenta derived fibroblast-like cell expresses KGF-receptor.

Preferred methods include embodiments wherein the culture solution is DMEM/F-12.

Preferred methods include embodiments wherein the culture solution further comprises a serum replacement agent.

Preferred methods include embodiments wherein the culture solution further comprises fetal calf serum.

Preferred methods include embodiments wherein the culture solution further comprises human serum.

Preferred methods include embodiments wherein the culture solution further comprises umbilical cord serum.

Preferred methods include embodiments wherein the culture solution further comprises platelet rich plasma.

Preferred methods include embodiments wherein a stem cell is generated by nuclear reprogramming of a mammalian somatic cell, the method comprising: providing a population of mammalian somatic cells comprising an endogenous pluripotency factor gene with: a. a first nucleic acid encoding from 2 to 7 distinct guide RNAs (gRNAs), each guide RNA comprising a DNA-binding segment and a polypeptide-binding segment, wherein the DNA-binding segment binds the promoter region of the endogenous pluripotency factor gene; and b. a second nucleic acid encoding at least one transcriptional modulator which binds the polypeptide-binding segment of the gRNAs, wherein the transcriptional modulator comprises an enzymatically inactive Cas9 polypeptide (dCas9), wherein the dCas9 is fused to a transcriptional activation domain; and culturing the mammalian somatic cells for a period of from about 2 to about 14 days, under conditions sufficient to (i) reprogram the mammalian somatic cell to an induced pluripotent stem cell (iPSC), and/or (ii) transdifferentiate the mammalian somatic cell to a target cell different in cell type from said mammalian somatic cell.

Preferred methods include embodiments wherein said pluripotency factor gene is oct3/4.

Preferred methods include embodiments wherein said pluripotency factor gene is sox2.

Preferred methods include embodiments wherein said pluripotency factor gene is klf4.

Preferred methods include embodiments wherein said pluripotency factor gene is c-myc.

Preferred methods include embodiments wherein said pluripotency factor gene is 1 in 28.

Preferred methods include embodiments wherein said pluripotency factor gene is nanog.

Preferred methods include embodiments wherein said pluripotency factor gene is glis-1.

Preferred methods include embodiments wherein said pluripotency factor gene is bcl2.

Preferred methods include embodiments wherein said pluripotency factor gene is bclx.

Preferred methods include embodiments wherein said pluripotency factor gene is livin.

Preferred methods include embodiments wherein said pluripotency factor gene is survivin.

Preferred methods include embodiments wherein said pluripotency factor gene is bxlsl.

Preferred methods include embodiments wherein said pluripotency factor gene is AIRE.

Preferred methods include embodiments wherein said pluripotency factor gene is HIF-1 alpha.

Preferred methods include embodiments wherein said pluripotency factor gene is one or more genes selectedfrom the group consisting of oct3/4, sox2, klf4, c-myc, lin28, nanog, glis-1, bcl2, bclxl, AIRE, HIF-1 alpha, survivin, livin and bclx.

Preferred methods include embodiments wherein, mammalian somatic cells is further provided with: a. a third nucleic acid encoding from 2 to 7 distinct gRNAs, each gRNA comprising a DNA-binding segment and a polypeptide-binding segment, wherein the DNA-binding segment binds the promoter region of a second endogenous pluripotency factor gene; and b. a fourth nucleic acid encoding from 2 to 7 distinct gRNAs, each gRNA comprising a DNA-binding segment and a polypeptide-binding segment, wherein the DNA-binding segment binds the promoter region of a third endogenous pluripotency factor gene; wherein the transcriptional modulator binds the polypeptide-binding segment of the gRNAs encoded by the third and fourth nucleic acids.

Preferred methods include embodiments wherein: (i) the DNA-binding segment of each the gRNAs encoded by the first nucleic acid is complementary to at least a portion of the promoter region of a mammalian oct3/4 gene; (ii) the DNA-binding segment of each the gRNAs encoded by the third nucleic acid is complementary to at least a portion of the promoter region of a mammalian sox2 gene; and (iii) the DNA-binding segment of each the gRNAs encoded by the fourth nucleic acid is complementary to at least a portion of the promoter region of a mammalian klf4 gene.

Preferred methods include embodiments wherein said pluripotent stem cell is transfected with a tumor antigen in order to induce immunity towards said tumor antigen.

Preferred methods include embodiments wherein said tumor antigen is CTCFL.

Preferred methods include embodiments wherein said tumor antigen is Fos-related antigen 1.

Preferred methods include embodiments wherein said tumor antigen is LCK.

Preferred methods include embodiments wherein said tumor antigen is FAP.

Preferred methods include embodiments wherein said tumor antigen is VEGFR2.

Preferred methods include embodiments wherein said tumor antigen is Fos-related antigen 1.

Preferred methods include embodiments wherein said tumor antigen is NA17.

Preferred methods include embodiments wherein said tumor antigen is PDGFR-beta.

Preferred methods include embodiments wherein said tumor antigen is PAP.

Preferred methods include embodiments wherein said tumor antigen is MAD-CT-2.

Preferred methods include embodiments wherein said tumor antigen is Tie-2.

Preferred methods include embodiments wherein said tumor antigen is PSA.

Preferred methods include embodiments wherein said tumor antigen is protamine.

Preferred methods include embodiments wherein said tumor antigen is legumain.

Preferred methods include embodiments wherein said tumor antigen is endosialin.

Preferred methods include embodiments wherein said tumor antigen is PSMA.

Preferred methods include embodiments wherein said tumor antigen is carbonic anhydrase IX.

Preferred methods include embodiments wherein said tumor antigen is STn.

Preferred methods include embodiments wherein said tumor antigen is Page4.

Preferred methods include embodiments wherein said tumor antigen is proteinase 3.

Preferred methods include embodiments wherein said tumor antigen is Fos-related antigen 1.

Preferred methods include embodiments wherein said tumor antigen is GM3 ganglioside.

Preferred methods include embodiments wherein said tumor antigen is tyrosinase.

Preferred methods include embodiments wherein said tumor antigen is MART1.

Preferred methods include embodiments wherein said tumor antigen is gp100.

Preferred methods include embodiments wherein said tumor antigen is SART3.

Preferred methods include embodiments wherein said tumor antigen is RGS5.

Preferred methods include embodiments wherein said tumor antigen is SSX2.

Preferred methods include embodiments wherein said tumor antigen is Globol1.

Preferred methods include embodiments wherein said tumor antigen is Tn.

Preferred methods include embodiments wherein said tumor antigen is CEA.

Preferred methods include embodiments wherein said tumor antigen is hCG.

Preferred methods include embodiments wherein said tumor antigen is PRAME.

Preferred methods include embodiments wherein said tumor antigen is XAGE.

Preferred methods include embodiments wherein said tumor antigen is AKAP-4.

Preferred methods include embodiments wherein said tumor antigen is TRP-2.

Preferred methods include embodiments wherein said tumor antigen is B7H3.

Preferred methods include embodiments wherein said tumor antigen is sperm fibrous sheath protein.

Preferred methods include embodiments wherein said tumor antigen is CYP1B1.

Preferred methods include embodiments wherein said tumor antigen is HMWMAA.

Preferred methods include embodiments wherein said tumor antigen is sLe.

Preferred methods include embodiments wherein said tumor antigen is MAGE A1.

Preferred methods include embodiments wherein said tumor antigen is PSMA.

Preferred methods include embodiments wherein said tumor antigen is GD2.

Preferred methods include embodiments wherein said tumor antigen is mesothelin.

Preferred methods include embodiments wherein said tumor antigen is fucosyl GM1.

Preferred methods include embodiments wherein said tumor antigen is GD3.

Preferred methods include embodiments wherein said tumor antigen is sperm protein 17.

Preferred methods include embodiments wherein said tumor antigen is NY-ESO-1.

Preferred methods include embodiments wherein said tumor antigen is PAX5.

Preferred methods include embodiments wherein said tumor antigen is AFP.

Preferred methods include embodiments wherein said tumor antigen is polysialic acid.

Preferred methods include embodiments wherein said tumor antigen is EpCAM.

Preferred methods include embodiments wherein said tumor antigen is MAGE-A3.

Preferred methods include embodiments wherein said tumor antigen is mutant p53.

Preferred methods include embodiments wherein said tumor antigen is ras.

Preferred methods include embodiments wherein said tumor antigen is mutant ras.

Preferred methods include embodiments wherein said tumor antigen is NY-BR1.

Preferred methods include embodiments wherein said tumor antigen is PAX-3.

Preferred methods include embodiments wherein said tumor antigen is Her2/neu.

Preferred methods include embodiments wherein said tumor antigen is OY-TES1.

Preferred methods include embodiments wherein said tumor antigen is HPV E6.

Preferred methods include embodiments wherein said tumor antigen is HPV E7.

Preferred methods include embodiments wherein said tumor antigen is PLAC1.

Preferred methods include embodiments wherein said tumor antigen is hTERT.

Preferred methods include embodiments wherein said tumor antigen is ML-IAP.

Preferred methods include embodiments wherein said tumor antigen is idiotype of b cell lymphoma or multiple myeloma.

Preferred methods include embodiments wherein said tumor antigen is EphA2.

Preferred methods include embodiments wherein said tumor antigen is EGFRvIII.

Preferred methods include embodiments wherein said tumor antigen is EGFRvIII.

### BRIEF DESCRIPTION OF THE DRAWINGS

FIG. 1 is a bar graph showing tumor growth in mice based on the following four treatments: 1) saline (Control), 2) unmodified stem cell derived DC (Unmodified), 3) stem cell derived DC expressing BORIS (BORIS) and 4) BORIS transfected DC with VEGF-R silenced (BORIS VEGFR Silenced).

### DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value, or the variation that exists among the study subjects. Typically the term is meant to encompass approximately or less than 1%, 2%, 3%, 4%, 5%, 6%, 7%, 8%, 9%, 10%, 11%, 12%, 13%, 14%, 15%, 16%, 17%,

18%, 19% or 20% variability depending on the situation.

The use of the term “or” in the claims is used to mean “and/or” unless explicitly indicated to refer to alternatives only or the alternatives are mutually exclusive, although the disclosure supports a definition that refers to only alternatives and “and/or.”

“Allogeneic,” as used herein, refers to cells of the same species that differ genetically from cells of a host.

“Autologous,” as used herein, refers to cells derived from the same subject. The term “engraft” as used herein refers to the process of stem cell incorporation into a tissue of interest in vivo through contact with existing cells of the tissue.

“Approximately” or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%,

2%, 1%, or less in either direction (greater than or less than) of the stated reference value unless otherwise stated or otherwise evident from the context (except where such number would exceed 100% of a possible value).

“Comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the current disclosure, and vice versa. Furthermore, compositions of the current disclosure can be used to achieve methods of the current disclosure.

“Somatic cell” it is meant any cell in an organism that has differentiated sufficiently, so that in the absence of experimental manipulation, does not ordinarily give rise to cells of all three germ layers of the body, i.e., ectoderm, mesoderm and endoderm. “Somatic cell” includes “multipotent cells” (i.e., progenitor cells), but does not include “pluripotent” or “totipotent cells.” For example, somatic cells would include both neurons and neural progenitors, the latter of which may be able to naturally give rise to all or some cell types of the central nervous system but cannot give rise to cells of the mesoderm or endoderm lineages.

“Multipotency” is referred to herein in the context of multipotent progenitor cells which have the potential to give rise to multiple cell types, but are less potent (more limited in their differentiation potential) than a pluripotent stem cell. For example, a multipotent stem cell is a hematopoietic cell that can develop into several types of blood cells, but cannot develop into brain cells or other types of cells.

“Pluripotent” is referred to herein as the property of a cell/cell type as having the potential to differentiate into any of the three germ layers: endoderm (e.g., interior stomach lining, gastrointestinal tract, the lungs), mesoderm (e.g., muscle, bone, blood, urogenital), or ectoderm (e.g., epidermal tissues and nervous system).

“Pluripotent stem cells” include natural pluripotent stem cells and induced pluripotent stem cells. They can give rise to any fetal or adult cell type. However, alone they generally cannot develop into a fetal or adult organism because they lack the potential to contribute to extra-embryonic tissue, such as the placenta.

“Induced pluripotent stem cells” or (“iPSCs”) are similar to natural pluripotent stem cells, such as embryonic stem cells, in many aspects, such as the expression of certain stem cell genes and/or proteins, chromatin methylation patterns, doubling time, embryoid body formation, teratoma formation, viable chimera formation, and potency and differentiability. Induced pluripotent cells may be derived from for example, adult stomach, liver, skin cells and blood cells (e.g., cord blood cells). iPSCs may be derived by transfection of synthetic transcription factors and/or certain stem cell-associated genes into non-pluripotent (e.g., somatic) cells. In certain embodiments, transfection may be achieved through viral vectors, such as retroviruses, for example, and non-viral or episomal vectors. Transfected genes can include, but are not limited to, reprogramming factors Oct3/4 (Pou5f1), Klf-4, c-Myc, Sox-2, Nanog and Lin28. Subpopulations of transfected cells may begin to become morphologically and biochemically similar to pluripotent stem cells, and can be isolated through morphological selection, doubling time, or through a reporter gene and antibiotic selection.

“Peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma. The term “leukemia” is meant broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyticleukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemi.

The term “carcinoma” refers to a malignant new growth made up of epithelial cells tending to infiltrate the surrounding tissues, and/or resist physiological and non-physiological cell death signals and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melano sarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some particular embodiments of the disclosure, the cancer treated is a melanoma. The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma. In one embodiment, stressed cells are recognized by the modified immune cells of the disclosure. Stressed cells may be recognized by immune cells that selectively are activated in response to proteins associated with stress such as heat shock proteins. Conditions that the disclosure may be useful for diagnosing are selected from the group consisting of: motor-neuron disease, multiple sclerosis, degenerative diseases of the CNS, dementia, Alzheimer's Disease, Parkinson's Disease, cerebrovascular accidents, epilepsy, temporary ischaemic accidents, mood disorders, psychotic illness, specific lobe dysfunction, pressure related CNS injury, cognitive dysfunction, deafness, blindness, anosmia, motor deficits, sensory deficits, head injury, trauma to the CNS, arrhythmias, myocardial infarction, pericarditis, congestive heart disease, valve related pathologies, myocardial dysfunction, endocardial dysfunction, pericardial dysfunction, sclerosis and thickening of valve flaps, fibrosis of cardiac muscle, decline in cardiac reserve, congenital defects of the heart or circulatory system, developmental defects of the heart or circulatory system, hypoxic or necrotic damage, blood vessel damage, cardiovascular disease (for example, angina, dissected aorta, thrombotic damage, aneurysm, atherosclerosis, emboli damage), disorders of the sweat gland, disorders of the sebaceous gland, piloerectile dysfunction, follicular problems, hair loss, epidermal disease, disease of the dermis or hypodermis, burns, ulcers, sores, infections, striae, seborrhoea, rosacea, disorders of the musculoskeletal system including disease and damage to muscles and bones, endocondral ossification, osteoporosis, osteomalacia, rickets, pagets disease, rheumatism, arthritis, diseases of the endocrine system, diseases of the lymphatic system, diseases of the urinary system, diseases of the reproductive system, metabolic diseases, diseases of the sinus, diseases of the nasopharynx, diseases of the oropharynx, diseases of the laryngopharynx, diseases of the larynx, diseases of the ligaments, diseases of the vocal cords, vestibular folds, glottis, epiglottis, trachea, mucocilliary mucosa, trachealis muscles, emphysema, chronic bronchitis, pulmonary infection, asthma, tuberculosis, cystic fibrosis, diseases of gas exchange, burns, barotraumas, dental care, periodontal disease, deglutination problems, ulcers, enzymatic disturbances/deficiencies, fertility problems, paralysis, dysfunction of absorption or absorptive services, diverticulosis, inflammatory bowel disease, hepatitis, cirrhosis, portal hypertension, diseases of sight, and cancer.

As used herein, “immune cell” refers to a cell capable of interacting with a cell that has abnormal qualities. Immune cells may include cells classically known to play a role in the immune system, such as T cells, B cells, and NK cells, or cells that are not classically considered immune cells but play a role in the identification of pathology. The cells include mesenchymal stem cells, hematopoietic stem cells or progeny thereof, and monocytes. In some embodiments immune cells are autologous to the recipient, or in other embodiments immune cells are allogeneic. In some specific embodiments, allogeneic cells are used that possess reduced allogenicity. Immune cells can include, for example, B cells, T cells, innate lymphoid cells, natural killer cells, natural killer T cells, gamma delta T cells, T regulatory cells, macrophages, monocytes, dendritic cells, neutrophils, myeloid derived suppressor cells.

“Redirected immune cell” refers to an immune cell, which has been modified to specifically recognize characteristics associated with an abnormal cell. In one specific example a “redirected immune cell” refers to a CAR-T cell, in another specific embodiment a “redirected immune cell refers to a cell made to express a non-endogenous receptor, wherein the non-endogenous receptor allows for a specific interaction with an abnormal cell.

As used herein, the term “population of immune cells” refers to one or more immune cells, such as a group of immune cells.

“Obtaining a population of immune cells” can be achieved by removal of a sample from a subject and purifying the population of immune cells. The population of immune cells may be obtained, for example, by obtaining a sample having a population of immune cells, including a blood sample, a tissue sample, or a biological fluid sample. The sample may be obtained by withdrawing blood or biological fluid from a subject or by removal of cells, tumors, or tissues from a subject.

“Delivery vehicle” refers to a molecule or composition useful for holding or suspending and transporting a compound in vivo for the purpose of localization and detection or release and delivery of the transported compound. Delivery vehicles can include, for example, B cells, T cells, innate lymphoid cells, natural killer cells, natural killer T cells, gamma delta T cells, T regulatory cells, macrophages, monocytes, dendritic cells, neutrophils, myeloid derived suppressor cells, mast cells, hematopoietic stem cells, fibroblasts, stromal vascular fraction, exosomes, endothelial progenitor cells, mesenchymal stem cells, pluripotent cell lines, or engineered nanoparticles. The delivery vehicle may be obtained by manufacture or by removal of a sample from a subject and purifying a delivery vehicle from the sample.

“Binding” or “interaction” as used herein (e.g. with reference to a synthetic transcriptional modulator binding the polypeptide-binding segment of a guide RNA) refers to a non-covalent interaction between macromolecules (e.g., between DNA and RNA, or between a polypeptide and a polynucleotide). “Binding” may also be referred to as “associated with” or “interacting”. “Binding” as used herein means that the binding partners are capable of binding to each other (e.g., will not necessarily bind to each other). Some portions of a binding interaction may be sequence-specific, but not all components of a binding interaction need be sequence-specific (e.g., contacts with phosphate residues in a DNA backbone). Binding interactions are generally characterized by a dissociation constant (Kd), e.g., less than 1 mM, less than 100 uM, less than 10 uM, less than 1 uM, less than 100 nM, less than 10 nM. “Affinity” refers to the strength of binding, increased binding affinity being correlated with a lower Kd.

“Promoter,” “promoter sequence,” or promoter region” refers to a DNA regulatory region/sequence capable of binding RNA polymerase and involved in initiating transcription of a downstream coding or non-coding sequence. In some examples of the present disclosure, the promoter sequence includes the transcription initiation site and extends upstream to include the minimum number of bases or elements necessary to initiate transcription at levels detectable above background. In some embodiments, the promoter sequence includes a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present invention.

“Vector” or “expression vector” is a replicon, such as a plasmid, phage, virus, or cosmid, to which another DNA segment, i.e. an “insert”, may be attached so as to bring about the replication of the attached DNA segment in a cell. “Vector” includes episomal (e.g., plasmids) and non episomal vectors. In some embodiments of the present disclosure the vector is an episomal vector, which is removed/lost from a population of cells after a number of cellular generations, e.g., by asymmetric partitioning.

“Expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression.

“Recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

“Efficiency of reprogramming” or “reprogramming efficiency” may be used to refer to the ability of cells to give rise to iPS cell colonies, e.g., when contacted with the synthetic transcription factors of the current disclosure. Somatic cells that demonstrate an enhanced efficiency of reprogramming to pluripotency will demonstrate an enhanced ability to give rise to iPSCs relative to a control. The term “efficiency of reprogramming” may also refer to the ability of somatic cells to be reprogrammed to a substantially different somatic cell type, a process known as transdifferentiation. The efficiency of reprogramming using the methods of the current disclosure vary with the particular combination of somatic cells, method of introducing synthetic transcription factors or reprogramming factors, and culturing methods following induction of reprogramming. The methods of the current disclosure may include “measuring reprogramming efficiency.” Determining the reprogramming efficiency can involve counting iPSC colonies, or may include measuring the expression of pluripotency markers, such as the below “key pluripotency markers” by the reprogrammed cells.

“Key pluripotency markers” known by one of ordinary skill in the art include but are not limited to the gene and/or protein expression of alkaline phosphatase, SSEA3, SSEA4, Sox2, Oct3/4, Nanog, TRA160, TRA181, TDGF 1, Dnmt3b, FoxD3, GDF3, Cyp26a1, TERT, and zfp42.

“Treating” or “treatment” is referred to herein as administration of a substance (e.g., pharmaceutical composition of the present disclosure) to a subject with the purpose to cure, alleviate, relieve, remedy, prevent, or ameliorate a disease or disorder, symptoms of the disorder, a disease state secondary to the disorder, or predisposition toward the disorder. An “effective amount” is an amount of the substance that is capable of producing a medically desirable result as delineated herein in a treated subject. The medically desirable result may be objective (i.e., measurable by some test or marker) or subjective (i.e., subject gives an indication of or feels an effect).

“Patient” as used herein refers to a mammalian subject diagnosed with or suspected of having or developing a disease amenable to stem cell therapy, e.g., cardiovascular disease. Exemplary patients may be humans, apes, dogs, pigs, cattle, cats, horses, goats, sheep, rodents and other mammals that can benefit from stem cell therapies.

“Administering” is referred to herein as providing the iPSCs of the current disclosure to a patient, e.g., by injection. By way of example and not limitation, administration may be performed by intravenous (i.v.) injection, sub-cutaneous (s.c.) injection, intradermal (i.d.) injection, intraperitoneal (i.p.) injection, or intramuscular (i.m.) injection. One or more such routes may be employed. Parenteral administration can be, for example, by bolus injection or by gradual perfusion over time. Alternatively, or concurrently, administration may be by the oral route. Additionally, administration may also be by surgical deposition of a bolus or pellet of cells, or positioning of a medical device, e.g., a stent, loaded with cells. Preferably, the compositions of the invention are administered at the site of disease, e.g. at the site or near (e.g., about or at least 1, 2, 3, 4, 5, 6, 7, 8, 9, 10, 11, 12, 13, 14, 15, 16, 17, 18, 19, 20, 25, 30, 40, 50 millimeters from) the site of a disease lesion (e.g., vascular stenosis/blockage, necrotic tissue or site of gangrenous infection).

“A patient in need thereof” is referred to herein as a patient diagnosed with or suspected of having disease amendable to stem cell therapy.

“Pluripotency factor gene” or “reprogramming factor gene” as used herein means an endogenous cellular gene encoding a pluripotency factor polypeptide (including its promoter region). Activation or repression of the expression of a pluripotency factor gene contributes to the nuclear reprogramming of a somatic cell, e.g., to multipotency or pluripotency. “Pluripotency factor gene” includes any target gene useful in the methods of the invention. Exemplary pluripotency factor genes include ESC-associated genes, such as reprogramming factor genes (which are typically activated in the methods of the present disclosure), and genes involved in initiating apoptosis (which are typically suppressed in the methods of the present disclosure).

“Pluripotency factor” or “reprogramming factor,” as used herein, refers to the corresponding gene product of the above “pluripotency factor gene” or “reprogramming factor gene.”

“Candidate pluripotency factor gene” refers to a gene potentially involved in nuclear reprogramming of a mammalian somatic cell, which is identified using the in vitro screening methods of the current disclosure utilizing candidate guide RNA (e.g., a library of candidate guide RNAs). Activation or repression of the expression of such gene results in the formation of iPSCs, e.g., the formation of at least one iPSC colony when undergoing an appropriate reprogramming procedure as outlined herein. The formation of an iPSC can indicate that a candidate guide RNA has hybridized to the promoter region of the candidate gene, and has targeted a transcriptional modulator to the regulatory region of the candidate gene. Subsequently, expression of the candidate gene has been modulated, thus potentially contributing to the reprogramming of the host cell. Identification of the “candidate pluripotency factor gene” may further involve matching the DNA-binding sequence of the candidate guide RNA with an endogenous gene sequence. Involvement of the candidate gene in reprogramming can be further verified, e.g., by repeating reprogramming of mammalian somatic cells using additional candidate gRNAs having the identified DNA-binding segment in combination with one or more transcriptional modulators of the present disclosure.

For the purpose of the invention, it is understood that reprogramming factor genes include POU5F1 (oct3/4), sox2, klf4, c-myc, lin28, and nanog. In some examples, the reprogramming factor genes being activated are at least two of oct3/4, sox-2, klf-4, c-myc, lin28, and nanog. In some examples, the reprogramming factor genes being activated are at least two of oct3/4, sox2, lin28, and nanog. In still other examples, the reprogramming factor genes are at least two of oct3/4, sox2, c-myc, and klf4. In other examples, the reprogramming factor genes being activated are at least three of oct3/4, sox2, lin28, and nanog. In still other examples, the reprogramming factor genes are at least three of oct3/4, sox2, c-myc, and klf4. In some examples, the reprogramming factor genes being activated are oct3/4, sox2, lin28, and nanog. In still other examples, the reprogramming factor genes being activated are oct3/4, sox-2, c-myc, and klf4. For generation of stem cells, it is disclosed in the invention that anti-apoptotic factors may be utilized. Said antiapoptotic factors include anti-apoptotic gene, for example bcl-2 or bcl-x. In some examples, the reprogramming factor genes being activated are at least two of oct3/4, sox-2, klf-4, c-myc, lin28, and nanog, and at least one anti-apoptotic gene (e.g., at least one of bcl-2 and bcl-x). In other examples, the reprogramming factor genes being activated are at least two of oct3/4, sox2, lin28, and nanog, and at least one anti-apoptotic gene (e.g., at least one of bcl-2 and bcl-x). In still other examples, the reprogramming factor genes being activated are at least two of oct3/4, sox2, c-myc, and klf4, and at least one anti-apoptotic gene (e.g., at least one of bcl-2 and bcl-x). In some examples, the reprogramming factor genes being activated are at least three of oct3/4, sox-2, lin28, and nanog, and at least one anti-apoptotic gene (e.g., at least one of bcl-2 and bcl-x). In still other examples, the reprogramming factor genes are at least three of oct3/4, sox2, c-myc, and klf4, and at least one anti-apoptotic gene (e.g., at least one of bcl-2 and bcl-x). In some examples, the reprogramming factor genes being activated are oct3/4, sox2, lin28, and nanog, and at least one anti-apoptotic gene (e.g., at least one of bcl-2 and bcl-x). In still other examples, the reprogramming factor genes being activated are oct3/4, sox-2, c-myc, and klf4, and at least one anti-apoptotic gene (e.g., at least one of bcl-2 and bcl-x).

It is to be understood that there are numerous ways of achieving cellular reprogramming or “dedifferentiation” of somatic cells to stem cells. Cellular reprogramming is traditionally accomplished using a combination of transcription factors (e.g., Oct3/4, Sox2, Klf4, Nanog, c-Myc and Lin28) [15-22], as well as genes that encode for proteins functioning as apoptotic repressors. Examples for these genes are SV-40 Large T-Antigen and the dominant negative form of the tumor suppressor protein, p53. Because genes for these apoptotic repressors do not reside endogenously in the human cell genome, in the CRIPR approach, apoptotic pathways that might be activated during the process of cellular reprogramming should be suppressed. Thus, in further examples according to any of the above embodiments, cellular reprogramming involves repression of at least one target gene, e.g., in combination with any one of the above described gene activations. In some examples, the target gene being repressed is an apoptosis promoting gene or a cell cycle inhibitor. Examples include p53 and its target gene p21, a cell cycle inhibitor. Repressing other cell cycle inhibitors could counteract apoptosis pathways triggered by the cellular reprogramming process. Some candidates are p19.sup.Arf (which stabilizes p53) and p16.sup. Ink4a (which prevents pRb from being phosphorylated by Cyclin D, and therefore induces cell cycle arrest). The Ink4/Arf locus is epigenetically silenced in iPSC, but upregulated in somatic cells, suggesting an important role of the Ink4a/Arf locus as an epigenetic barrier to reprogramming. Thus, in some examples, the target gene being repressed is selected from p53, p21, p19.sup.Arf, and p16.sup. Ink4a. The silencing and/or gene editing of Ink4A/Arf is supported by numerous studies. In one study, investigators described that the overexpression of defined transcription factors in somatic cells results in their reprogramming into induced pluripotent stem (iPS) cells. The extremely low efficiency and slow kinetics of in vitro reprogramming suggest that further rare events are required to generate iPS cells. The nature and identity of these events, however, remain elusive. The investigators noticed that the reprogramming potential of primary murine fibroblasts into iPS cells decreases after serial passaging and the concomitant onset of senescence. Consistent with the notion that loss of replicative potential provides a barrier for reprogramming, here they showed that cells with low endogenous p19(Arf) (encoded by the Ink4a/Arf locus, also known as Cdkn2a locus) protein levels and immortal fibroblasts deficient in components of the Arf-Trp53 pathway yield iPS cell colonies with up to threefold faster kinetics and at a significantly higher efficiency than wild-type cells, endowing almost every somatic cell with the potential to form iPS cells. Notably, the acute genetic ablation of Trp53 (also known as p53) in cellular subpopulations that normally fail to reprogram rescues their ability to produce iPS cells. These results show that the acquisition of immortality is a crucial and rate-limiting step towards the establishment of a pluripotent state in somatic cells [23]

The invention provides other examples according to any of the above embodiments, the pluripotency factor gene being repressed is a gene encoding for signal transduction proteins that promote cell death and/or cell cycle arrest. Examples include Rho-associated protein kinase (ROCK), and kinases belonging to the AGC (PKA/PKG/PKC) family of serine-threonine kinases. ROCK is mainly involved in regulating the shape and movement of cells by acting on the cytoskeleton. ROCK inhibition has been shown to promote cell survival of pluripotent stem cells as single cells, by preventing dissociation-induced apoptosis. Moreover, repressing ROCK will potentially inhibit the mTOR pathway. Inhibition of the mTOR pathway by rapamycin, for example, notably enhances the reprogramming efficiency (T. Chen, L. Shen, J. Yu et al., “Rapamycin and other longevity promoting compounds enhance the generation of mouse induced pluripotent stem cells,” Aging Cell 2011, 10(5):908-911). Thus, in some examples, the pluripotency factor gene being repressed is selected from ROCK, a PKA/PKG/PKC family kinase, and other genes who's repression would inhibit the mTOR pathway.

Another pluripotency factor gene useful in the methods of the invention is glis1. In some embodiments of the present disclosure, mammalian somatic cells are contacted with an exogenous reprogramming factor. Exogenous reprogramming factors are provided to the cell as compositions of isolated polypeptides, i.e. in a biologically active cell-free form, or as exogenous nucleic acids (e.g., DNA, RNA) encoding the same, which upon delivery to the cell or upon expression, reprogram or contribute to reprogramming a somatic cell to, e.g., multipotency or pluripotency. In some embodiments, the reprogramming factors may be non-integrating, i.e., provided to the recipient somatic cell in a form that does not result in integration of exogenous DNA into the genome of the recipient cell.

Biological activity may be determined by specific DNA binding assays; or by determining the effectiveness of the factor in altering cellular transcription. A composition of the invention may provide one or more biologically active reprogramming factors. The composition may comprise at least about 50.mu.g/ml soluble reprogramming factor, at least about 100.mu.g/ml; at least about 150.mu.g/ml, at least about 200.mu.g/ml, at least about 250.mu.g/ml, at least about 300.mu.g/ml, or at least about 500 ug/ml.

A Klf4 polypeptide is a polypeptide comprising the amino acid sequence that is at least 70% identical to the amino acid sequence of human Klf4, i.e., Kruppel-Like Factor 4 the sequence of which may be found at GenBank Accession Nos. NP 004226 and NM 004235. Klf4 polypeptides, e.g. those that are at least 70%, 75%, 80%, 85%, 90%, 91%, 92%, 95%, 97%, 99%, or 100% identical to the sequence provided in GenBank Accession No. NM 004235, and the nucleic acids that encode them find use as a reprogramming factor in the present invention.

A c-Myc polypeptide is a polypeptide comprising an amino acid sequence that is at least 70% identical to the amino acid sequence of human c-Myc, i.e., myelocytomatosis viral oncogene homolog, the sequence of which may be found at GenBank Accession Nos. NP_002458 and NM002467, c-Myc polypeptides, e.g. those that are at least 70%, 75%, 80%, 85%, 90%, 91%, 92%, 95%, 97%, 99%, or 100% identical to the sequence provided in GenBank Accession No. NM 002467, and the nucleic acids that encode them find use as a reprogramming factor in the present invention.

A Nanog polypeptide is a polypeptide comprising an amino acid sequence that is at least 70% identical to the amino acid sequence of human Nanog, i.e., Nanog homeobox, the sequence of which may be found at GenBank Accession Nos. NP_079141 and NM 024865.

Nanog polypeptides, e.g. those that are at least 70%, 75%, 80%, 85%, 90%, 91%, 92%, 95%, 97%, 99%, or 100% identical to the sequence provided in GenBank Accession No. NM 024865 and the nucleic acids that encode them find use as a reprogramming factor in the present invention.

A Lin-28 polypeptide is a polypeptide comprising an amino acid sequence that is at least 70% identical to the amino acid sequence of human Lin-28, i.e., Lin-28 homolog of C. elegans, the sequence of which may be found at GenBank Accession Nos. NP_078950 and NM 024674. Lin-28 polypeptides, e.g. those that are at least 70%, 75%, 80%, 85%, 90%, 91%, 92%, 95%, 97%, 99%, or 100% identical to the sequence provided in GenBank Accession No. NM 024674, and the nucleic acids that encode them find use as a reprogramming factor in the present invention.

An Oct3/4 polypeptide is a polypeptide comprising an amino acid sequence that is at least 70% identical to the amino acid sequence of human Oct3/4, also known as Homo sapiens POU class 5homeobox 1 (POU5F1) the sequence of which may be found at GenBank Accession Nos. NP_002692 and NM 002701. Oct3/4 polypeptides, e.g. those that are at least 70%, 75%, 80%, 85%, 90%, 91%, 92%, 95%, 97%, 99%, or 100% identical to the sequence provided in GenBank Accession No. NM 002701, and the nucleic acids that encode them find use as a reprogramming factor in the present invention.

A Sox2 polypeptide is a polypeptide comprising the amino acid sequence at least 70% identical to the amino acid sequence of human Sox2, i.e., sex-determining region Y-box 2 protein, the sequence of which may be found at GenBank Accession Nos. NP_003097 and NM 003106. Sox2 polypeptides, e.g. those that are at least 70%, 75%, 80%, 85%, 90%, 91%, 92%, 95%, 97%, 99%, or 100% identical to the sequence provided in GenBank Accession No. NM 003106, and the nucleic acids that encode them find use as a reprogramming factor in the present invention.

The methods of the current disclosure may also include contacting the mammalian somatic cell with a small molecule or reprogramming enhancer that can alter or modulate transcription. In some examples, the small molecule or reprogramming enhancer is a histone deacetylase (HDAC) inhibitor. Small molecules, including without limitation siRNAs/miRNAs [24-38], valproic acid, hydroxamic acid, trichostatin A, suberoylanilide hydroxamic acid, BIX-01294 and BayK8644 have been described as useful in reprogramming cells. Other reprogramming enhancers useful in the methods of the current disclosure include aluminum-containing salts (e.g., aluminum hydroxide) and TGF-beta inhibitors (e.g., A83-01).

In one embodiment embodiments, the invention utilizes pluripotent stem cells such as inducible pluripotent stem cells, multipotent progenitor cells and/or progenitor cells committed to specific hematopoietic lineages as starting cells. The hematopoietic progenitor cells may be derived from a tissue such as bone marrow, peripheral blood (including mobilized peripheral blood), umbilical cord blood, placental blood, aortal-gonadal-mesonephros derived cells, and lymphoid soft tissue. Lymphoid soft tissue includes the thymus, spleen, liver, lymph node, skin, tonsil and/or Peyer's patches. In other embodiments, the lymph reticular stromal cells may be also derived from at least one of the foregoing lymphoid soft tissues. In preferred embodiments, the hematopoietic stem cells are generated from inducible pluripotent stem cells, and said cells are differentiated into the myeloid lineage. In some embodiments the cells are generatically altered in order to endow to said cells anticancer properties.

In one embodiment of the invention, stem cells are generated through somatic cell nuclear transfer [39, 40] or parthenogenesis [41]. In some embodiments of the invention, reprogramming factors are used to reprogram somatic cells to pluripotency. Said reprogramming factors may include cytoplasm or extracts from a pluripotent stem cell or oocyte [42-47], extracellular vesicles from pluripotent stem cells [48], fusion with pluripotent stem cells [49-55], or chemically defined factors such as Oct3/4 [56-58], Sox2, Lin28, and Nanog [59-61]. These are transcription factors that maintain pluripotency in embryonic stem (ES) cells while Klf4 [62], and c-Myc are transcription factors thought to boost iPSC generation efficiency [63-66]. The transcription factor c-Myc is believed to modify chromatin structure to allow Oct3/4 and Sox2 to more efficiently access genes necessary for reprogramming while Klf4 enhances the activation of certain genes by Oct3/4 and Sox2. Nanog, like Oct3/4 and Sox2, is a transcription factor that maintains pluripotency in ES cells while Lin28 is an mRNA-binding protein thought to influence the translation or stability of specific mRNAs during differentiation. It has also been shown that retroviral expression of Oct3/4 and Sox2, together with co-administration of valproic acid, a chromatin destabilizer and histone deacetylase inhibitor, is sufficient to reprogram fibroblasts into iPSCs. Various small molecules can also be used to induce dedifferentiation as described [67-78]. All of these methods are useful within the practice of the invention for generation of inducible pluripotent stem cells.

In some embodiments the invention teaches the generation of not just dendritic cells with genetically altered features but also other antigen presenting cells. Antigen presenting cells include cells such as dendritic cells, monocytes/macrophages, Langerhans cells, Kupfer cells, microglia, alveolar macrophages and B cells. In other embodiments, the antigen presenting cells are derived from hematopoietic progenitor cells in vitro. Various embodiments are provided, wherein the hematopoietic progenitor cells, the lymph reticular stromal cells, and porous solid matrix. The antigen presenting cells may be derived from hematopoietic progenitor cells in vitro. In important embodiments the antigen presenting cells are mature. In further embodiments, the method Jo further comprises administering a co-stimulatory agent to the co-culture. In another embodiment a solid porous matrix is provided wherein hematopoietic progenitor cells, with or without their progeny, and lymph reticular stromal cells are attached to the solid porous matrix. The lymph reticular stromal cells are present in an amount sufficient to support the growth and differentiation of hematopoietic progenitor cells.

In certain embodiments, the hematopoietic progenitor cells are attached to the lymph reticular stromal cells. In further embodiments, the solid porous matrix may include antigen presenting cells (progeny and/or nonprogeny). Preferably the antigen presenting cells are mature. In yet further embodiments, the porous matrix further comprises at least one antigen. The porous matrix can be one that is an open cell porous matrix having a percent open space of at least 50%, and preferably at least 75%. In one embodiment the porous solid matrix has pores defined by interconnecting ligaments having a diameter at midpoint, on average, of less than micrometers. Preferably the porous solid matrix is a metal-coated reticulated open cell foam of carbon containing material, the metal coating being selected from the group consisting of tantalum, titanium, platinum (including other metals of the platinum group), niobium, hafnium, tungsten, and combinations thereof. In preferred embodiments, whether the porous solid matrix is metal-coated or not, the matrix is coated with a biological agent selected from the group consisting of collagens, fibronectins, laminins, integrins, angiogenic factors, anti-inflammatory factors, glycosaminoglycans, vitrogen, antibodies and fragments thereof, functional equivalents of these factors, and combinations thereof. Most preferably the metal coating is tantalum coated with a biological agent. In certain other embodiments the porous solid matrix having seeded hematopoietic progenitor cells and lymph reticular stromal cells, is impregnated with a gelatinous agent that occupies pores of the matrix.

In a further aspect of the invention, a method for identifying an agent suspected of affecting hematopoietic cell development, is provided. The method involves introducing an amount of hematopoietic progenitor cells and an amount of lymph reticular stromal cells into a porous, solid matrix having interconnected pores of a pore size sufficient to permit the hematopoietic progenitor cells and the lymphoreticular stromal cells to grow throughout the matrix, co-culturing the hematopoietic progenitor cells and the lymphoreticular stromal cells in the presence of at least one candidate agent suspected of affecting hematopoietic cell development (in a test co-culture), and determining whether the at least one candidate agent affects hematopoietic cell development in the test co-culture by comparing the test co-culture hematopoietic cell development to a control co-culture, whereby hematopoietic progenitor cells and lymphoreticular stromal cells are co-cultured in the absence of the at least one candidate agent.

Various embodiments are provided, wherein the hematopoietic progenitor cells, the lymphoreticular stromal cells, and the porous solid matrix have one or more of the preferred characteristics as described above, and the cells are cultured as described above. In certain embodiments, hematopoietic progenitor cell development includes hematopoietic progenitor cell maintenance, expansion, differentiation toward a specific cell lineage, and/or cell-death (including apoptosis). In preferred embodiments the lymphoreticular stromal cells are thymic stromal cells.

For the practice of the invention, pluripotent stem cells are differentiated into dendritic cells. In one embodiment, said dendritic cells are pulsed with antigen and/or gene modified before administration. In other embodiments a stem cell bank is generated from pluripotent stem cells, and said pluripotent stem cells are transfected with antigens and/or gene silenced. Gene silencing of said stem cells is performed to make differentiated progeny of stem cells resistant to cancer induced inhibitory effects on dendritic cells. This allows for said dendritic cells to be implanted in a manner so that they are resistant to tumor secreted dendritic cell inhibiting activities. Tumors are known to secrete a variety of factors that inhibit dendritic cell maturation and/or function. In some studies, VEGF has been shown to inhibit dendritic cell maturation.

Differentiation of dendritic cells from pluripotent cells may be performed using various means known in the art. In one embodiment, a 3 step protocol for efficient neutrophil production from hiPSCs in 2D serum- and feeder-free conditions was developed using direct programming with modified mRNA (mmRNA). Initially, hiPSCs are directly programmed into hematoendothelial progenitors using ETV2 mmRNA which then differentiated into myeloid progenitors in the presence of GM-CSF, FGF2 and UM171. Non-adherent myeloid progenitors can be continuously collected from cultures every 8-10 days for up to 30 days post ETV2 transfection, and subsequently differentiated into mature neutrophils in the presence of G-CSF and a retinoic acid agonist, for example, Am580. This method significantly expedites generation of neutrophils with the first batch of neutrophils available as soon as 14 days after initiation of differentiation and allows the generation of up to 1.7.times.10.sup. 7 neutrophils from 10.sup. 6 hPSCs. Further, these in vitro derived neutrophils are genetically manipulatable, which is not true for donor derived neutrophils, and therefore can be genetically altered relative to conditions needed for the specific treatments.

In one aspect, the disclosure provides an in vitro method of producing CD16.sup.+CD10.sup.-neutrophils from pluripotent stem cells (PSCs) in serum-free medium, the method comprising: (a)transiently introducing exogenous ETV2 in the PSCs and culturing the ETV2-induced PSCs in xenogen-free serum-free medium comprising FGF-2 to produce a population of ETV2-induced CD144.sup.+hematoendothelial progenitor cells (ETV2-induced HEPs); (b) culturing the ETV2-induced CD144.sup.+thematoendothelial progenitor cells in xenogen-free serum-free medium comprising GM-CSF and FGF2 for a sufficient time to produce non-adherent myeloid progenitors; and (c) culturing the non-adherent myeloid progenitors in xenogen-free serum-free medium comprising G-CSF and retinoic acid agonist to differentiate the myeloid progenitors into neutrophils.

In yet another aspect, the disclosure provides an in vitro method of producing CD14. sup.+CD16. sup.+macrophages from pluripotent stem cells (PSCs), the method comprising:

(a) transiently introducing exogenous ETV2 into pluripotent stem cells and culturing the ETV2-induced PSCs in xenogen- and serum-free medium comprising FGF2 to produce ETV2-induced CD144.sup.+hematoendothelial progenitor cells;

(b) culturing the ETV2-induced CD144.sup.+hematoendothelial progenitor cells in xenogen- and serum-free medium comprising GM-CSF and FGF-2 for about 4 days; and

(c) culturing the cells of step (b) in medium comprising IL-3 and M-CSF to produce the CD14.sup. 30 CD16.sup.+macrophages expressing CD68, CD80 and CD163.

In yet a further aspect, the disclosure provides an in vitro derived CD14.sup.+CD16.sup.+macrophage population made by the methods described herein, wherein the macrophages further express CD68, CD80 and CD163. The foregoing and other aspects and advantages of the invention will appear from the following description. In the description, reference is made to the accompanying drawings which form a part hereof, and in which there are shown, by way of illustration, preferred embodiments of the invention. Such embodiments do not necessarily represent the full scope of the invention, however, and reference is made therefore to the claims and herein for interpreting the scope of the invention.

Numerous animal models have demonstrated that in the context of neoplasia DCs can bind to and engulf tumour antigens that are released from tumor cells, either alive or dying, and cross-present these antigens to T cells in tumour-draining lymph nodes. This results in the generation of tumour-specific immune responses that have been demonstrated to inhibit tumor growth or in some cases induced transferrable immunological memory. Mechanistically, DCs recognize tumors using the same molecular means that they would use to recognize apoptotic cells, or cells that are stressed. One set of signals are molecules released from apoptotic cells, which are highly released by tumors, these include the nucleotides UTP and ATP, fractalkine, lipid lysophosphatidylcholine, and sphingosine 1-phosphate [79]. Signals from stressed cells, such as tumor cells include externalization of phosphatidylserine onto the outside of the cell membrane, calreticulin, avß5 integrin, CD36 and lactadherin. There is some evidence that dendritic cells actively promote tumor immunity in that patients with dendritic cell infiltration of tumors generally have a better prognosis [80-83].

For the practice of the invention, dendritic cells generated from pluripotent stem cells can be used for treatment of cancer according to previously published protocols. In one embodiment, dendritic cells are generated lacking molecules associated with cancer induced immune suppression. Said molecules include receptors and/or downstream signaling molecules of immune suppressive substances. In one embodiment pluripotent stem cells are generated by gene editing and/or gene silencing of molecules such as VEGF receptor, TGF-beta receptor, IL-10 receptor, IL-13 receptor, adenosine receptor. Downstream signaling molecules include members of the SMAD family, ITIMs, and phosphatases. In some cases dendritic cell immunotherapy protocols are utilized from existing immunotherapy protocols and incorporated by reference. Therapeutic activities of DC have been demonstrated in melanoma [84-135], soft tissue sarcoma [136], thyroid [137-139], glioma [140-161], multiple myeloma,[162-170], lymphoma [171-173], leukemia [174-181], as

well as liver [182-187], lung [188-201], ovarian [202-205], and pancreatic cancer [206-208].

The generation of DC immunotherapies from pluripotent stem cells can be used in a manner that conventional DC would be used. There are numerous descriptions of prior art that can be used to teach one of skill in the art to teach the practice of the invention. We provide some information on uses of dendritic cells. While DC themselves are part of the initial immune response to cancer, numerous mechanisms are used by tumors to suppress the ability of DC to stimulate an immune response. One particular mechanism is the depletion of tryptophan in the tumor microenvironment by production of indolamine 2,3 deoxygenase (IDO) [209], which will be discussed in detail in Section 5.6. Tryptophan depletion results in T cell apoptosis, while catabolites of tryptophan depletion are known to lead to suppression of T cell activation. In order to overcome issues associated with local tumor suppression of DC, numerous studies have utilized ex vivo generated DC that have been pulsed with tumor antigen as a means of stimulating anticancer immunity. In some embodiments, IDO silencing in pluripotent iPSC cells is performed, in another embodiment, IDO is gene edited.

The most advanced DC based therapy is the product Provenge (sipuleucel-T), which is approved by the FDA for treatment of androgen resistant prostate cancer. Provenge is a cellular product derived from autologous peripheral blood mononuclear cell (PBMC) derived dendritic cells that have been grown using a chimeric protein comprised of GM-CSF and the prostate specific antigen, prostatic acid phosphatase [210, 211]. In the pivotal trial, this DC based therapeutic resulted in extension of survival by 4.1 months [211]. Prior to approval of Provenge, numerous clinical trials using DC were performed in prostate cancer, which will be discussed below.

Tjoa et al reported on 33 participants of a phase I trial in patients with advanced prostate cancer that received autologous DC pulsed HLA-A0201-specific prostate-specific membrane antigen (PSMA) peptides (PSM-P1 or -P2) that were entered into a second trial (Phase II) which involved six infusions of DC pulsed with PSM-P1 and -P2 peptides. The patients were followed up for up to 770 days from the start of the original phase I study. 9 partial responders were identified in the phase II study based on National Prostate Cancer Project (NPCP) criteria, plus 50% reduction of prostate-specific antigen. Four of the partial responders were also responders in the phase I study, with an average response duration of 225 days. Their combined average total response period was over 370 days. Five other responders in the secondary immunizations at the Phase II were nonresponders in the phase I study. Their average partial response period was 196 days. These data support the safety of follow-up infusion of DC that have been pulsed with tumor antigen derived peptide [212]. The same group published a subsequent paper on an additional 33 patients that had not received prior DC immunization in the Phase I. All subjects received six infusions of DC pulsed with PSM-P1 and -P2 at six week intervals without any treatment associated adverse events. Six partial and two complete responders were identified in the phase II study based on NPCP criteria, plus 50% reduction of prostate-specific antigen (PSA), or resolution in previously measurable lesions on ProstaScint scan [213]. The same group analyzed immune response in patients who had clinical remission or relapsed. A strong correlation was found between delayed type hypersensitivity response to the PSM-P1 and PSM-P2 and clinical response [214].

The approach that was to evolve into Provenge was described in a paper that reported outcome of 12 androgen resistant prostate cancer patients treated with DC that were pulsed with a GM-CSF-PAP fusion protein. Two intravenous infusions of the generated cells were performed one month apart. The infusions were followed by three s.c. monthly doses of the fusion protein without cells. Treatment was well tolerated and circulating prostate-specific antigen levels dropped in three patients. Immune response to the fusion protein was observed, as well as to PAP [215]. A subsequent study utilized the Provenge approach as used today, that is, without the subcutaneous boosting with protein alone. In this study, DC precursors were harvested by leukapheresis in weeks 0, 4, 8, and 24, loaded ex vivo with antigen for 2 days, and then infused intravenously over 30 minutes. Phase I patients received increasing doses of Provenge, and phase II patients received all the Provenge that could be prepared from a leukapheresis product. Patients tolerated treatment well. Fever, the most common adverse event, occurred after 15 infusions (14.7%). All patients developed immune responses to the recombinant fusion protein used to prepare Provenge, and 38% developed immune responses to PAP. Three patients had a more than 50% decline in prostate-specific antigen (PSA) level, and another three patients had 25% to 49% decreases in PSA. The time to disease progression correlated with development of an immune response to PAP and with the dose of dendritic cells received [216]. An additional study utilized the same approach to treat 2l patients with histologically documented androgen-independent prostate carcinoma that could be evaluated by radionuclide bone scan or computed tomography scan. Provenge was prepared from a leukapheresis product; it contained autologous CD54-positive recombinant GM-CSF-PAP loaded DC with admixtures of monocytes, macrophages, B and T cells. Provenge was infused intravenously twice, 2 weeks apart. Two weeks after the second infusion, patients received three subcutaneous injections of 1.0 mg of the recombinant protein 1 month apart. Nineteen patients could be evaluated for response to treatment. The median time to progression was 118 days. Treatment was tolerated reasonably well; most adverse effects were secondary to Provenge and were NCI Common Toxicity Criteria Grade 1-2. Four of the 21 patients reported Grade 3-4 adverse events. Two patients exhibited a transient 25-50% decrease in prostate-specific antigen (PSA). Fora third patient, PSA dropped from 221 ng/ml at baseline to undetectable levels by week 24 and has remained so for more than 4 years. In addition, this patient's metastatic retroperitoneal and pelvic adenopathy has resolved. PBMC collected from patients for at least 16 weeks proliferated upon in vitro stimulation by the recombinant GM-CSF-PAP. For the patient with responsive disease, PBMC could be stimulated for 96 weeks [217]. Another study assessed the effects of Provenge on androgen independent prostate cancer with biochemical progression. This type of cancer is earlier in the oncogenesis process as compared to androgen resistant cancer. Specifically, patients with no metastatic recurrent disease as manifested by increasing PSA levels (0.4-6.0 ng/mL) and who had undergone previous definitive surgical or radiation therapy were enrolled. Therapy consisted of Provenge infusions on weeks 0, 2, and 4 (ie, 3 infusions). Prostate-specific antigen was measured at baseline and monthly until disease progression, defined as a doubling of the baseline or nadir PSA value (whichever was lower) to > or =4 ng/mL or development of distant metastases. Thirteen of 18 patients demonstrated an increase in PSA doubling time (PSADT), with a median increase of 62% (4.9 months before treatment vs. 7.9 months after treatment; P=0.09; signed-rank test). These data suggested that Provenge has therapeutic activity both on androgen dependent, which is more early stage, and androgen dependent, which is more late stage, prostate cancer [218]. The Phase III trial for Provenge consisted of 512 randomly assigned prostate cancer patients in a 2:1 ratio to receive either Provenge (341 patients) or placebo (171 patients) administered intravenously every 2 weeks, for a total of three infusions. The primary end point was overall survival, analyzed by means of a stratified Cox regression model adjusted for baseline levels of serum prostate-specific antigen (PSA) and lactate dehydrogenase. In the Provenge group, there was a relative reduction of 22% in the risk of death as compared with the placebo group (P=0.03). This reduction represented a 4.1-month improvement in median survival (25.8 months in the Provenge group vs. 21.7 months in the placebo group). The 36-month survival probability was 31.7% in the Provenge group versus 23.0% in the placebo group. Immune responses to the immunizing antigen were observed in patients who received Provenge but not controls [219].

In addition to Provenge, which as mentioned above, received FDA marketing approval, several other types of antigens have been utilized in DC therapy of prostate cancer. For example, while PSA is a known biochemical marker of prostate cancer progression, the PSA protein or peptides from this protein have been identified to possess immunogenic properties. One study examined the possibility of utilizing PSA protein pulsed DC for treatment of prostate cancer. Twenty-four patients with histologically proven prostate carcinoma and an isolated postoperative rise of serum PSA (>1 ng/ml to 10 ng/ml) after radical prostatectomy were included. The patients received nine administrations of PSA-loaded DCs by combined intravenous, subcutaneous, and intradermal routes over 21 weeks. Circulating prostate cancer cells detected in six patients at baseline were undetectable at 6 months and remained undetectable at 12 months. Eleven patients had a post baseline transient PSA decrease on one to three occasions, predominantly occurring at month 1 (7 patients) or month 3 (2 patients). Maximum PSA decrease ranged from 6% to 39%. PSA decrease on at least one occasion was more frequent in patients with low Gleason score (p=0.016) at prostatectomy and with positive skin tests at study baseline (p=0.04). PSA-specific T cells were detected ex vivo by ELISpot for IFN-gamma in 7 patients before vaccination and in 11 patients after vaccination. Of the latter 11 patients, 5 had detectable T cells both before and during the vaccination period, 4 only during the vaccination period, while 2 patients could for technical reasons not be assessed prevaccination. No induction of anti-PSA IgG or IgM antibodies was detected. There were no serious adverse events or otherwise severe toxicities observed during the trial [220].

Proteins may possess both immune stimulatory and immune inhibitory epitopes. Thus some studies sought to utilize specific peptides which are known to be immune stimulatory. Accordingly, a clinical trial was conducted in 28 patients with locally advanced or metastatic prostate cancer to determine whether an HLA-A2 binding epitope of prostate-specific antigen, PSA 146-154 (PSA-peptide), can induce specific T cell immunity. Patients were vaccinated either by intradermal injection of PSA-peptide and GM-CSF or by intravenous administration of autologous dendritic cells pulsed with PSA-peptide at weeks 1, 4 and 10. DTH skin testing was performed at weeks 4, 14, 26 and 52. Fifty percent of the patients developed positive DTH responses to PSA-peptide. Cytokine analysis of PSA-peptide stimulated T cells exhibited specific IFN-gamma and TNF-alpha response in six of seven patients. Specific IL-4 response was observed in five patients, while IL-10 response was detected in one patient. Purified CD4-CD8+ T cells isolated from four patients demonstrated specific cytolytic activity per chromium release assay. This trial demonstrated that, immunization with PSA-peptide induced specific T cell immunity in one-half of the patients with locally advanced and hormone-sensitive, metastatic prostate cancer. DTH-derived T cells exhibited PSA-peptide-specific cytolytic activity and predominantly expressed a type-1 cytokine profile [221]. A subsequent study sought to boost effects of PSA peptide pulsed DC through administration of interferon gamma in the treatment of 12 hormone resistant prostate cancer patients. All patients were vaccinated four times with intracutaneously injected PSA-peptide loaded DCs after subcutaneous administration of IFN-gamma 2 hr before DC administration (50 microg/m(2) body surface). The vaccination was well tolerated without any vaccination-associated adverse events. One partial and one mixed responder were identified, four patients showed stable diseases. Two patients had a decrease and four a slow-down velocity slope in the PSA serum level. All responders showed a positive DTH-response, but only two a slight increase in PSA-peptide specific T-lymphocytes [222].

Given that tumors may suppress expression of certain peptides, or alternatively may mutate the peptide, a more global approach towards stimulation of anticancer immunity has been the utilization of multiple peptides to overcome this hurdles. A clinical study in 8 androgen resistant prostate cancer patients utilized a cocktail consisting of HLA-A*0201-restricted peptides derived from five different prostate cancer-associated antigens [prostate-specific antigen (PSA), prostate-specific membrane antigen (PSMA), survivin, prostein, transient receptor potential p8 (trp-p8)]. Patients were treated with 4 vaccinations of pulsed DC once every two weeks. Apart from local skin reactions no side effects were noted. One patient displayed a partial response (PR; PSA decrease >50%) and three other patients showed stable PSA values or decelerated PSA increases. In ELISPOT analyses, three of four PSA responders also showed antigen-specific CD8+ T-cell activation against prostein, survivin, and PSMA [223]. Cocktail approaches have been used by other investigators using different peptides, for example, a study by Waeckerle-Men et al. utilized autologous DC of HLA-A*0201(+) patients with hormone-refractory prostate cancer that were loaded with antigenic peptides derived from prostate stem cell antigen (PSCA(14-22)), prostatic acid phosphatase (PAP(299-307)), prostate-specific membrane antigen (PSMA(4-12)), and prostate-specific antigen (PSA(154-163)). DC were intradermally applied six times at biweekly intervals followed-in the case of an enhanced immune response-by monthly booster injections. Of the three patients that were reported, the vaccination elicited significant cytotoxic T cell responses against all prostate-specific antigens tested. In addition, memory T cell responses against the control peptides derived from influenza matrix protein and tetanus toxoid were efficiently boosted. Clinically, the long-term DC vaccination was associated with an increase in PSA doubling time [224].

### Example 1: iPS Derived Dendritic Cells Induce Reduction of B16 Melanoma

iPSCs (ATCC-DYR0100 Human Induced Pluripotent Stem (IPS) Cells (ATCC® ACS-1011™) were cultured on feeder layers of OP9 cells for 6 to 7 days in α-MEM supplemented with 20% FBS. The mesodermally differentiated cells were then harvested, reseeded onto fresh OP9 cell layers, and cultured in α-MEM supplemented with 20% FBS, 20 ng/mL GM-CSF, and 50 μmol/L2-ME. On day 13 to 14, floating cells were recovered by pipetting. These cells were considered to be iPSC-derived myeloid cells (iPS-MCs). The cells were infected with lentivirus vectors expressing the c-Myc and the Brother of the Regulatory of Imprinted Sites (BORIS) gene, as well as shRNA encoding siRNA silencing VEGF-R in the presence of 8 ng/mL polybrene (Sigma-Aldrich), and were cultured in α-MEM supplemented with 20% FBS, 30 ng/mL GM-CSF, and 30 ng/mL M-CSF. After 5 to 6 days, proliferating cells appeared and were considered to be ESC- or iPSC-derived pMCs (ES-pMC or iPS-pMC, respectively). To induce the differentiation of these cells into DC-like cells (pMC-DC), they were cultured in RPMI-1640 supplemented with 20% FBS in the presence of 20 ng/mL IL4 plus 30 ng/mL GM-CSF for 3 days.

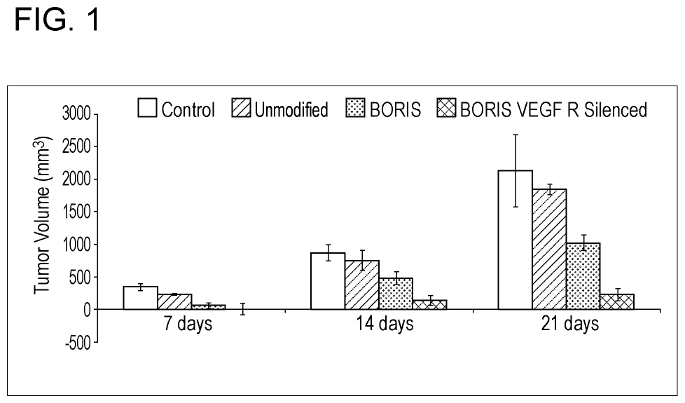

Mice were inoculated with 500,000 B16 melanoma. Mice were injected with saline (Control), unmodified stem cell derived DC (Unmodified), stem cell derived DC expressing BORIS (BORIS) and BORIS transfected DC with VEGF-R silenced (BORIS VEGF R Silenced). Tumor growth was assessed by calipers. Results are shown in FIG. 1.

### REFERENCES

1. Steinman, R. M. and Z. A. Cohn, Identification of a novel cell type in peripheral lymphoid organs of mice. I. Morphology, quantitation, tissue distribution. J Exp Med, 1973. 137(5): p. 1142-62.
2. Banchereau, J. and R. M. Steinman, Dendritic cells and the control of immunity. Nature, 1998. 392(6673): p. 245-52.
3. Trombetta, E. S. and I. Mellman, Cell biology of antigen processing in vitro and in vivo. Annu Rev Immunol, 2005. 23: p. 975-1028.
4. Itano, A. A. and M. K. Jenkins, Antigen presentation to naive CD4 T cells in the lymphnode. Nat Immunol, 2003. 4(8): p. 733-9.
5. Caux, C., et al., Activation of human dendritic cells through CD40 cross-linking. J ExpMed, 1994. 180(4): p. 1263-72.
6. Pulendran, B., K. Palucka, and J. Banchereau, Sensing pathogens and tuning immune responses. Science, 2001. 293(5528): p. 253-6.
7. Lutz, M. B., et al., Culture of bone marrow cells in GM-CSF plus high doses of lipopolysaccharide generates exclusively immature dendritic cells which induce alloantigen-specific CD4 T cell anergy in vitro. Eur J Immunol, 2000. 30(4): p. 1048-52.
8. Turnquist, H. R. and A. W. Thomson, Taming the lions: manipulating dendritic cells for use as negative cellular vaccines in organ transplantation. Curr Opin Organ Transplant, 2008. 13(4): p. 350-7.
9. Pletinckx, K., et al., Role of dendritic cell maturity/costimulation for generation, homeostasis, and suppressive activity of regulatory T cells. Front Immunol, 2011. 2: p. 39.
10. Frasca, L., et al., Human anergic CD4+ T cells can act as suppressor cells by affecting autologous dendritic cell conditioning and survival. J Immunol, 2002. 168(3): p. 1060-8.
11. Vendetti, S., et al., Anergic T cells inhibit the antigen presenting function of dendritic cells. J Immunol, 2000. 165(3): p. 1175-81.
12. Veldhoen, M., et al., Modulation of dendritic cell function by naive and regulatory CD4+ T cells. J Immunol, 2006. 176(10): p. 6202-10.
13. Guillot, C., et al., Active suppression of allogeneic proliferative responses by dendritic cells after induction of long-term allograft survival by CTLA4Ig. Blood, 2003. 101(8): p. 3325-33.
14. Roelofs-Haarhuis, K., et al., Infectious nickel tolerance: a reciprocal interplay of tolerogenic APCs and T suppressor cells that is driven by immunization. J Immunol, 2003. 171(6): p. 2863-72.
15. Maherali, N., et al., A high-efficiency system for the generation and study of human induced pluripotent stem cells. Cell Stem Cell, 2008. 3(3): p. 340-5.
16. Okita, K., et al., Generation of mouse induced pluripotent stem cells without viral vectors. Science, 2008. 322(5903): p. 949-53.
17. Darr, H. and N. Benvenisty, Genetic analysis of the role of the reprogramming gene LIN-28 in human embryonic stem cells. Stem Cells, 2009. 27(2): p. 352-62.
18. Choi, K. D., et al., Hematopoietic and endothelial differentiation of human induced pluripotent stem cells. Stem Cells, 2009. 27(3): p. 559-67.
19. Gonzalez, F., et al., Generation of mouse-induced pluripotent stem cells by transient expression of a single nonviral polycistronic vector. Proc Natl Acad Sci USA, 2009. 106(22): p. 8918-22.
20. Lu, L., et al., Pluripotent factor lin-28 and its homologue lin-28b in epithelial ovarian cancer and their associations with disease outcomes and expression of let-7a and IGF-II. Eur J Cancer, 2009. 45(12): p. 2212-8.
21. Utikal, J., et al., Sox2 is dispensable for the reprogramming of melanocytes and melanoma cells into induced pluripotent stem cells. J Cell Sci, 2009. 122(Pt 19): p. 3502-10.
22. Newman, M. A. and S. M. Hammond, Lin-28: an early embryonic sentinel that blocks Let-7 biogenesis. Int J Biochem Cell Biol, 2010. 42(8): p. 1330-3.
23. Utikal, J., et al., Immortalization eliminates a roadblock during cellular reprogramming into iPS cells. Nature, 2009. 460(7259): p. 1145-8.
24. Ma, Y., et al., High-efficiency siRNA-based gene knockdown in human embryonic stem cells. RNA, 2010. 16(12): p. 2564-9.
25. Rim, J. S., et al., Screening for epigenetic target genes that enhance reprogramming using lentiviral-delivered shRNA. Methods Mol Biol, 2011. 702: p. 299-316.
26. Li, Z., et al., Small RNA-mediated regulation of iPS cell generation. EMBO J, 2011. 30(5): p. 823-34.
27. Ding, L., et al., From RNAi screens to molecular function in embryonic stem cells. Stem Cell Rev Rep, 2012. 8(1): p. 32-42.
28. Ma, Y., H. Lin, and C. Qiu, High-efficiency transfection and siRNA-mediated gene knockdown in human pluripotent stem cells. Curr Protoc Stem Cell Biol, 2012. Chapter 2: p. Unit 5C 2.
29. Guo, X., et al., microRNA-29b is a novel mediator of Sox2 function in the regulation of somatic cell reprogramming. Cell Res, 2013. 23(1): p. 142-56.
30. Yang, S. H., et al., A genome-wide RNAi screen reveals MAP kinase phosphatases as key ERK pathway regulators during embryonic stem cell differentiation. PLoS Genet, 2012. 8(12): p. e1003112.
31. Staszkiewicz, J., et al., Silencing histone deacetylase-specific isoforms enhances expression of pluripotency genes in bovine fibroblasts. Cell Reprogram, 2013. 15(5): p. 397-404.
32. Fan, A., et al., Effects of TET1 knockdown on gene expression and DNA methylation in porcine induced pluripotent stem cells. Reproduction, 2013. 146(6): p. 569-79.
33. Chen, Y., et al., The miR-134 attenuates the expression of transcription factor FOXM1 during pluripotent NT2/D1 embryonal carcinoma cell differentiation. Exp Cell Res, 2015. 330(2): p. 442-450.
34. Choi, Y. J., et al., Deficiency of microRNA miR-34a expands cell fate potential in pluripotent stem cells. Science, 2017. 355(6325).
35. Dang, J. and T. M. Rana, Enhancing Induced Pluripotent Stem Cell Generation by MicroRNA. Methods Mol Biol, 2016. 1357: p. 71-84.
36. Li, N., et al., microRNAs: important regulators of stem cells. Stem Cell Res Ther, 2017. 8(1): p. 110.
37. Kulcenty, K., et al., MicroRNA Profiling During Neural Differentiation of Induced Pluripotent Stem Cells. Int J Mol Sci, 2019. 20(15).
38. Anokye-Danso, F., et al., Highly efficient miRNA-mediated reprogramming of mouse and human somatic cells to pluripotency. Cell Stem Cell, 2011. 8(4): p. 376-88.
39. Wernig, M., et al., In vitro reprogramming of fibroblasts into a pluripotent ES-cell-like state. Nature, 2007. 448(7151): p. 318-24.
40. Mitalipov, S. M., et al., Reprogramming following somatic cell nuclear transfer in primates is dependent upon nuclear remodeling. Hum Reprod, 2007. 22(8): p. 2232-42.
41. Ju, J. Y., et al., Establishment of stem cell lines from nuclear transferred and parthenogenetically activated mouse oocytes for therapeutic cloning. Fertil Steril, 2008.89 (5 Suppl): p. 1314-23.
42. Mitalipov, S. M., et al., Oct-4 expression in pluripotent cells of the rhesus monkey. Biol Reprod, 2003. 69(6): p. 1785-92.
43. Freberg, C. T., et al., Epigenetic reprogramming of OCT4 and NANOG regulatory regions by embryonal carcinoma cell extract. Mol Biol Cell, 2007. 18(5): p. 1543-53.
44. Miyamoto, K., et al., Reprogramming events of mammalian somatic cells induced by Xenopus laevis egg extracts. Mol Reprod Dev, 2007. 74(10): p. 1268-77.
45. Grinnell, K. L. and J. R. Bickenbach, Skin keratinocytes pre-treated with embryonic stem cell-conditioned medium or BMP4 can be directed to an alternative cell lineage. Cell Prolif, 2007. 40(5): p. 685-705.
46. Neri, T., et al., Mouse fibroblasts are reprogrammed to Oct-4 and Rex-1 gene expression and alkaline phosphatase activity by embryonic stem cell extracts. Cloning Stem Cells, 2007. 9(3): p. 394-406.
47. Rajasingh, J., et al., Cell free embryonic stem cell extract-mediated derivation of multipotent stem cells from NIH3T3 fibroblasts for functional and anatomical ischemic tissue repair. Circ Res, 2008. 102(11): p. e107-17.
48. Ratajczak, J., et al., Embryonic stem cell-derived microvesicles reprogram hematopoietic progenitors: evidence for horizontal transfer of mRNA and protein delivery. Leukemia, 2006. 20(5): p. 847-56.
49. Tada, M., et al., Nuclear reprogramming of somatic cells by in vitro hybridization with ES cells. Curr Biol, 2001. 11(19): p. 1553-8.
50. Munsie, M., C. O'Brien, and P. Mountford, Transgenic strategy for demonstrating nuclear reprogramming in the mouse. Cloning Stem Cells, 2002. 4(2): p. 121-30.
51. Hansis, C., et al., Nuclear reprogramming of human somatic cells by Xenopus egg extract requires BRG1. Curr Biol, 2004. 14(16): p. 1475-80.
52. Do, J. T. and H. R. Scholer, Nuclei of embryonic stem cells reprogram somatic cells. Stem Cells, 2004. 22(6): p. 941-9.
53. Yu, J., et al., Human embryonic stem cells reprogram myeloid precursors following cell-cell fusion. Stem Cells, 2006. 24(1): p. 168-76.
54. Do, J. T. and H. R. Scholer, Cell-cell fusion as a means to establish pluripotency. Ernst Schering Res Found Workshop, 2006(60): p. 35-45.
55. Ma, D. K., et al., G9a and Jhdm2a regulate embryonic stem cell fusion-induced reprogramming of adult neural stem cells. Stem Cells, 2008. 26(8): p. 2131-41.
56. Grinnell, K. L., et al., De-differentiation of mouse interfollicular keratinocytes by the embryonic transcription factor Oct-4. J Invest Dermatol, 2007. 127(2): p. 372-80.
57. Buitrago, W. and D. R. Roop, Oct-4: the almighty POUripotent regulator? J Invest Dermatol, 2007. 127(2): p. 260-2.
58. Lin, H., et al., Stem cell regulatory function mediated by expression of a novel mouse Oct4 pseudogene. Biochem Biophys Res Commun, 2007. 355(1): p. 111-6.
59. Meissner, A., M. Wernig, and R. Jaenisch, Direct reprogramming of genetically unmodified fibroblasts into pluripotent stem cells. Nat Biotechnol, 2007. 25(10): p. 1177-81.
60. Park, I. H., et al., Reprogramming of human somatic cells to pluripotency with defined factors. Nature, 2008. 451(7175): p. 141-6.
61. Levasseur, D. N., et al., Oct4 dependence of chromatin structure within the extended Nanog locus in ES cells. Genes Dev, 2008. 22(5): p. 575-80.
62. Jiang, J., et al., A core Klf circuitry regulates self-renewal of embryonic stem cells. Nat Cell Biol, 2008. 10(3): p. 353-60.
63. Yu, J., et al., Induced pluripotent stem cell lines derived from human somatic cells. Science, 2007. 318(5858): p. 1917-20.
64. Lowry, W. E., et al., Generation of human induced pluripotent stem cells from dermal fibroblasts. Proc Natl Acad Sci USA, 2008. 105(8): p. 2883-8.
65. Hanna, J., et al., Direct reprogramming of terminally differentiated mature B lymphocytes to pluripotency. Cell, 2008. 133(2): p. 250-64.
66. Schenke-Layland, K., et al., Reprogrammed mouse fibroblasts differentiate into cells of the cardiovascular and hematopoietic lineages. Stem Cells, 2008. 26(6): p. 1537-46.
67. Huangfu, D., et al., Induction of pluripotent stem cells by defined factors is greatly improved by small-molecule compounds. Nat Biotechnol, 2008. 26(7): p. 795-7.
68. Shi, Y., et al., Induction of pluripotent stem cells from mouse embryonic fibroblasts by Oct4 and Klf4 with small-molecule compounds. Cell Stem Cell, 2008. 3(5): p. 568-74.
69. Carpenedo, R. L., et al., Homogeneous and organized differentiation within embryoid bodies induced by microsphere-mediated delivery of small molecules. Biomaterials, 2009. 30(13): p. 2507-15.
70. Desponts, C. and S. Ding, Using small molecules to improve generation of induced pluripotent stem cells from somatic cells. Methods Mol Biol, 2010. 636: p. 207-18.
71. Li, Y., et al., Generation of iPSCs from mouse fibroblasts with a single gene, Oct4, and small molecules. Cell Res, 2011. 21(1): p. 196-204.
72. Yuan, X., et al., Brief report: combined chemical treatment enables Oct4-induced reprogramming from mouse embryonic fibroblasts. Stem Cells, 2011. 29(3): p. 549-53.
73. Krohne, T. U., et al., Generation of retinal pigment epithelial cells from small molecules and OCT4 reprogrammed human induced pluripotent stem cells. Stem Cells Transl Med, 2012. 1(2): p. 96-109.
74. Vendrell, M., et al., A fluorescent screening platform for the rapid evaluation of chemicals in cellular reprogramming. Stem Cell Res, 2012. 9(3): p. 185-91.
75. Li, W., et al., Identification of Oct4-activating compounds that enhance reprogramming efficiency. Proc Natl Acad Sci USA, 2012. 109(51): p. 20853-8.
76. Zhang, Z. and W. S. Wu, Sodium butyrate promotes generation of human induced pluripotent stem cells through induction of the miR302/367 cluster. Stem Cells Dev, 2013. 22(16): p. 2268-77.
77. Gross, B., et al., Improved generation of patient-specific induced pluripotent stem cells using a chemically-defined and matrigel-based approach. Curr Mol Med, 2013. 13(5): p. 765-76.
78. Feltes, B. C. and D. Bonatto, Combining small molecules for cell reprogramming through an interatomic analysis. Mol Biosyst, 2013. 9(11): p. 2741-63.
79. Ravichandran, K. S., Beginnings of a good apoptotic meal: the find-me and eat-me signaling pathways. Immunity, 2011. 35(4): p. 445-55.
80. Hu, M., et al., Decreased intratumoral Foxp3 Tregs and increased dendritic cell density by neoadjuvant chemotherapy associated with favorable prognosis in advanced gastric cancer. Int J Clin Exp Pathol, 2014. 7(8): p. 4685-94.
81. Ayari, C., et al., High level of mature tumor-infiltrating dendritic cells predicts progression to muscle invasion in bladder cancer. Hum Pathol, 2013. 44(8): p. 1630-7.
82. Liska, V., et al., Infiltration of colorectal carcinoma by S100+ dendritic cells and CD57+ lymphocytes as independent prognostic factors after radical surgical treatment. Anticancer Res, 2012. 32(5): p. 2129-32.
83. Ayari, C., et al., Bladder tumor infiltrating mature dendritic cells and macrophages as predictors of response to bacillus Calmette-Guerin immunotherapy. Eur Urol, 2009. 55(6): p. 1386-95.
84. Nestle, F. O., et al., Vaccination of melanoma patients with peptide-or tumor lysate-pulsed dendritic cells. Nat Med, 1998. 4(3): p. 328-32.
85. Chakraborty, N. G., et al., Immunization with a tumor-cell-lysate-loaded autologous-antigen-presenting-cell-based vaccine in melanoma. Cancer Immunol Immunother, 1998. 47(1): p. 58-64.
86. Wang, F., et al., Phase I trial of a MART-1 peptide vaccine with incomplete Freund's adjuvant for resected high-risk melanoma. Clin Cancer Res, 1999. 5(10): p. 2756-65.
87. Thurner, B., et al., Vaccination with mage-3A1 peptide-pulsed mature, monocyte-derived dendritic cells expands specific cytotoxic T cells and induces regression of some metastases in advanced stage IV melanoma. J Exp Med, 1999. 190(11): p. 1669-78.
88. Thomas, R., et al., Immature human monocyte-derived dendritic cells migrate rapidly to draining lymph nodes after intradermal injection for melanoma immunotherapy. Melanoma Res, 1999. 9(5): p. 474-81.
89. Mackensen, A., et al., Phase I study in melanoma patients of a vaccine with peptide-pulsed dendritic cells generated in vitro from CD34(+) hematopoietic progenitor cells. Int J Cancer, 2000. 86(3): p. 385-92.
90. Panelli, M. C., et al., Phase 1 study in patients with metastatic melanoma of immunization with dendritic cells presenting epitopes derived from the melanoma-associated antigens MART-1 and gp100. J Immunother, 2000. 23(4): p. 487-98.
91. Schuler-Thurner, B., et al., Mage-3 and influenza-matrix peptide-specific cytotoxic T cells are inducible in terminal stage HLA-A2.1+ melanoma patients by mature monocyte-derived dendritic cells. J Immunol, 2000. 165(6): p. 3492-6.
92. Lau, R., et al., Phase I trial of intravenous peptide pulsed dendritic cells in patients with metastatic melanoma. J Immunother, 2001. 24(1): p. 66-78.
93. Banchereau, J., et al., Immune and clinical responses in patients with metastatic melanoma to CD34(+) progenitor-derived dendritic cell vaccine. Cancer Res, 2001. 61(17): p. 6451-8.
94. Schuler-Thurner, B., et al., Rapid induction of tumor-specific type 1 T helper cells in metastatic melanoma patients by vaccination with mature, cryopreserved, peptide-loaded monocyte-derived dendritic cells. J Exp Med, 2002. 195(10): p. 1279-88.
95. Palucka, A. K., et al., Single injection of CD34+ progenitor-derived dendritic cell vaccine can lead to induction of T-cell immunity in patients with stage IV melanoma. J Immunother, 2003. 26(5): p. 432-9.
96. Bedrosian, I., et al., Intranodal administration of peptide-pulsed mature dendritic cell vaccines results in superior CD8+ T-cell function in melanoma patients. J Clin Oncol, 2003. 21(20): p. 3826-35.
97. Slingluff, C. L., Jr., et al., Clinical and immunologic results of a randomized phase II trial of vaccination using four melanoma peptides either administered in granulocyte-macrophage colony-stimulating factor in adjuvant or pulsed on dendritic cells. J Clin Oncol, 2003. 21(21): p. 4016-26.
98. Hersey, P., et al., Phase I/II study of treatment with dendritic cell vaccines in patients with disseminated melanoma. Cancer Immunol Immunother, 2004. 53(2): p. 125-34.
99. Vilella, R., et al., Pilot study of treatment of biochemotherapy-refractory stage IV melanoma patients with autologous dendritic cells pulsed with a heterologous melanoma cell line lysate. Cancer Immunol Immunother, 2004. 53(7): p. 651-8.
100. Palucka, A. K., et al., Spontaneous proliferation and type 2 cytokine secretion by CD4+ T cells in patients with metastatic melanoma vaccinated with antigen pulsed dendritic cells. J Clin Immunol, 2005. 25(3): p. 288-95.
101. Banchereau, J., et al., Immune and clinical outcomes in patients with stage IV melanoma vaccinated with peptide-pulsed dendritic cells derived from CD34+ progenitors and activated with type I interferon. J Immunother, 2005. 28(5): p. 505-16.
102. Trakatelli, M., et al., A new dendritic cell vaccine generated with interleukin-3 and interferon-beta induces CD8+ T cell responses against NA17-A2 tumor peptide in melanoma patients. Cancer Immunol Immunother, 2006. 55(4): p. 469-74.
103. Salcedo, M., et al., Vaccination of melanoma patients using dendritic cells loaded with an allogeneic tumor cell lysate. Cancer Immunol Immunother, 2006. 55(7): p. 819-29.
104. Linette, G. P., et al., Immunization using autologous dendritic cells pulsed with the melanoma-associated antigen gp100-derived G280-9V peptide elicits CD8+ immunity. Clin Cancer Res, 2005. 11(21): p. 7692-9.
105. Escobar, A., et al., Dendritic cell immunizations alone or combined with low doses of interleukin-2 induce specific immune responses in melanoma patients. Clin Exp Immunol, 2005. 142(3): p. 555-68.
106. Tuettenberg, A., et al., Induction of strong and persistent MelanA/MART-1-specific immune responses by adjuvant dendritic cell-based vaccination of stage II melanoma patients. Int J Cancer, 2006. 118(10): p. 2617-27.
107. Schadendorf, D., et al., Dacarbazine (DTIC) versus vaccination with autologous peptide-pulsed dendritic cells (DC) in first-line treatment of patients with metastatic melanoma: a randomized phase III trial of the DC study group of the DeCOG. Ann Oncol, 2006. 17(4): p. 563-70.
108. Di Pucchio, T., et al., Immunization of stage IV melanoma patients with Melan-A/MART-1 and gp100 peptides plus IFN-alpha results in the activation of specific CD8(+) T cells and monocyte/dendritic cell precursors. Cancer Res, 2006. 66(9): p. 4943-51.
109. Nakai, N., et al., Vaccination of Japanese patients with advanced melanoma with peptide, tumor lysate or both peptide and tumor lysate pulsed mature, monocyte-derived dendritic cells. J Dermatol, 2006. 33(7): p. 462-72.
110. Palucka, A. K., et al., Dendritic cells loaded with killed allogeneic melanoma cells can induce objective clinical responses and MART-1 specific CD8+ T-cell immunity. J Immunother, 2006. 29(5): p. 545-57.
111. Lesimple, T., et al., Immunologic and clinical effects of injecting mature peptide-loaded dendritic cells by intralymphatic and intranodal routes in metastatic melanoma patients. Clin Cancer Res, 2006. 12(24): p. 7380-8.
112. Guo, J., et al., Intratumoral injection of dendritic cells in combination with local hyperthermia induces systemic antitumor effect in patients with advanced melanoma. Int J Cancer, 2007. 120(11): p. 2418-25.
113. O'Rourke, M. G., et al., Dendritic cell immunotherapy for stage IV melanoma. MelanomaRes, 2007. 17(5): p. 316-22.
114. Bercovici, N., et al., Analysis and characterization of antitumor T-cell response after administration of dendritic cells loaded with allogeneic tumor lysate to metastatic melanoma patients. J Immunother, 2008. 31(1): p. 101-12.
115. Hersey, P., et al., Phase I/II study of treatment with matured dendritic cells with or without low dose IL-2 in patients with disseminated melanoma. Cancer Immunol Immunother, 2008. 57(7): p. 1039-51.
116. von Euw, E. M., et al., A phase I clinical study of vaccination of melanoma patients with dendritic cells loaded with allogeneic apoptotic/necrotic melanoma cells. Analysis of toxicity and immune response to the vaccine and of IL-10-1082 promoter genotype as predictor of disease progression. J Transl Med, 2008. 6: p. 6.
117. Carrasco, J., et al., Vaccination of a melanoma patient with mature dendritic cells pulsed with MAGE-3 peptides triggers the activity of nonvaccine anti-tumor cells. J Immunol, 2008. 180(5): p. 3585-93.
118. Redman, B. G., et al., Phase Ib trial assessing autologous, tumor-pulsed dendritic cells as a vaccine administered with or without IL-2 in patients with metastatic melanoma. J Immunother, 2008. 31(6): p. 591-8.
119. Daud, A. I., et al., Phenotypic and functional analysis of dendritic cells and clinical outcome in patients with high-risk melanoma treated with adjuvant granulocyte macrophage colony-stimulating factor. J Clin Oncol, 2008. 26(19): p. 3235-41.
120. Engell-Noerregaard, L., et al., Review of clinical studies on dendritic cell-based vaccination of patients with malignant melanoma: assessment of correlation between clinical response and vaccine parameters. Cancer Immunol Immunother, 2009. 58(1): p. 1-14.
121. Nakai, N., et al., Immunohistological analysis of peptide-induced delayed-type hypersensitivity in advanced melanoma patients treated with melanoma antigen-pulsed mature monocyte-derived dendritic cell vaccination. J Dermatol Sci, 2009. 53(1): p. 40-7.
122. Dillman, R. O., et al., Phase II trial of dendritic cells loaded with antigens from self-renewing, proliferating autologous tumor cells as patient-specific antitumor vaccines inpatients with metastatic melanoma: final report. Cancer Biother Radiopharm, 2009. 24(3): p. 311-9.
123. Chang, J. W., et al., Immunotherapy with dendritic cells pulsed by autologous dactinomycin-induced melanoma apoptotic bodies for patients with malignant melanoma. Melanoma Res, 2009. 19(5): p. 309-15.
124. Trepiakas, R., et al., Vaccination with autologous dendritic cells pulsed with multiple tumor antigens for treatment of patients with malignant melanoma: results from a phase I/II trial. Cytotherapy, 2010. 12(6): p. 721-34.
125. Jacobs, J. F., et al., Dendritic cell vaccination in combination with anti-CD25 monoclonal antibody treatment: a phase I/II study in metastatic melanoma patients. Clin Cancer Res, 2010. 16(20): p. 5067-78.
126. Ribas, A., et al., Multicenter phase II study of matured dendritic cells pulsed with melanoma cell line lysates in patients with advanced melanoma. J Transl Med, 2010. 8:p. 89.
127. Ridolfi, L., et al., Unexpected high response rate to traditional therapy after dendritic cell-based vaccine in advanced melanoma: update of clinical outcome and subgroup analysis. Clin Dev Immunol, 2010. 2010: p. 504979.
128. Cornforth, A. N., et al., Resistance to the proapoptotic effects of interferon-gamma on melanoma cells used in patient-specific dendritic cell immunotherapy is associated with improved overall survival. Cancer Immunol Immunother, 2011. 60(1): p. 123-31.
129. Lesterhuis, W. J., et al., Wild-type and modified gp100 peptide pulsed dendritic cell vaccination of advanced melanoma patients can lead to long-term clinical responses independent of the peptide used. Cancer Immunol Immunother, 2011. 60(2): p. 249-60.
130. Bjoern, J., et al., Changes in peripheral blood level of regulatory T cells in patients with malignant melanoma during treatment with dendritic cell vaccination and low-dose IL-2.Scand J Immunol, 2011. 73(3): p. 222-33.
131. Steele, J. C., et al., Phase I/II trial of a dendritic cell vaccine transfected with DNA encoding melan A and gp100 for patients with metastatic melanoma. Gene Ther, 2011. 18(6): p. 584-93.
132. Kim, D. S., et al., Immunotherapy of malignant melanoma with tumor lysate-pulsed autologous monocyte-derived dendritic cells. Yonsei Med J, 2011. 52(6): p. 990-8.
133. Ellebaek, E., et al., Metastatic melanoma patients treated with dendritic cell vaccination, Interleukin-2 and metronomic cyclophosphamide: results from a phase II trial. Cancer Immunol Immunother, 2012. 61(10): p. 1791-804.
134. Dillman, R. O., et al., Tumor stem cell antigens as consolidative active specific immunotherapy: a randomized phase II trial of dendritic cells versus tumor cells inpatients with metastatic melanoma. J Immunother, 2012. 35(8): p. 641-9.
135. Dannull, J., et al., Melanoma immunotherapy using mature DCs expressing the constitutive proteasome. J Clin Invest, 2013. 123(7): p. 3135-45.
136. Finkelstein, S. E., et al., Combination of external beam radiotherapy (EBRT) with intratumoral injection of dendritic cells as neo-adjuvant treatment of high-risk soft tissue sarcoma patients. Int J Radiat Oncol Biol Phys, 2012. 82(2): p. 924-32.
137. Stift, A., et al., Dendritic cell vaccination in medullary thyroid carcinoma. Clin CancerRes, 2004. 10(9): p. 2944-53.
138. Kuwabara, K., et al., Results of a phase I clinical study using dendritic cell vaccinations for thyroid cancer. Thyroid, 2007. 17(1): p. 53-8.
139. Bachleitner-Hofmann, T., et al., Pilot trial of autologous dendritic cells loaded with tumor lysate(s) from allogeneic tumor cell lines in patients with metastatic medullary thyroid carcinoma. Oncol Rep, 2009. 21(6): p. 1585-92.
140. Yu, J. S., et al., Vaccination of malignant glioma patients with peptide pulsed dendritic cells elicits systemic cytotoxicity and intracranial T-cell infiltration. Cancer Res, 2001. 61(3): p. 842-7.
141. Yamanaka, R., et al., Vaccination of recurrent glioma patients with tumour lysate-pulsed dendritic cells elicits immune responses: results of a clinical phase I/II trial. Br J Cancer, 2003. 89(7): p. 1172-9.
142. Yu, J. S., et al., Vaccination with tumor lysate-pulsed dendritic cells elicits antigen-specific, cytotoxic T-cells in patients with malignant glioma. Cancer Res, 2004. 64(14): p. 4973-9.
143. Yamanaka, R., et al., Tumor lysate and IL-18 loaded dendritic cells elicits Th1 response, tumor-specific CD8+ cytotoxic T cells in patients with malignant glioma. J Neurooncol, 2005. 72(2): p. 107-13.
144. Yamanaka, R., et al., Clinical evaluation of dendritic cell vaccination for patients with recurrent glioma: results of a clinical phase I/II trial. Clin Cancer Res, 2005. 11(11): p. 4160-7.
145. Liau, L. M., et al., Dendritic cell vaccination in glioblastoma patients induces systemic and intracranial T-cell responses modulated by the local central nervous system tumor microenvironment. Clin Cancer Res, 2005. 11(15): p. 5515-25.
146. Walker, D. G., et al., Results of a phase I dendritic cell vaccine trial for malignant astrocytoma: potential interaction with adjuvant chemotherapy. J Clin Neurosci, 2008. 15(2): p. 114-21.
147. Leplina, O. Y., et al., Use of interferon-alpha-induced dendritic cells in the therapy of patients with malignant brain gliomas. Bull Exp Biol Med, 2007. 143(4): p. 528-34.
148. De Vleeschouwer, S., et al., Postoperative adjuvant dendritic cell-based immunotherapy in patients with relapsed glioblastoma multiforme. Clin Cancer Res, 2008. 14(10): p. 3098-104.
149. Ardon, H., et al., Adjuvant dendritic cell-based tumour vaccination for children with malignant brain tumours. Pediatr Blood Cancer, 2010. 54(4): p. 519-25.
150. Prins, R. M., et al., Gene expression profile correlates with T-cell infiltration and relative survival in glioblastoma patients vaccinated with dendritic cell immunotherapy. Clin Cancer Res, 2011. 17(6): p. 1603-15.
151. Okada, H., et al., Induction of CD8+ T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. J Clin Oncol, 2011. 29(3): p. 330-6.
152. Fadul, C. E., et al., Immune response in patients with newly diagnosed glioblastoma multiforme treated with intranodal autologous tumor lysate-dendritic cell vaccination after radiation chemotherapy. J Immunother, 2011. 34(4): p. 382-9.
153. Chang, C. N., et al., A phase I/II clinical trial investigating the adverse and therapeutic effects of a postoperative autologous dendritic cell tumor vaccine in patients with malignant glioma. J Clin Neurosci, 2011. 18(8): p. 1048-54.
154. Cho, D. Y., et al., Adjuvant immunotherapy with whole-cell lysate dendritic cells vaccine for glioblastoma multiforme: a phase II clinical trial. World Neurosurg, 2012. 77(5-6): p. 736-44.
155. Iwami, K., et al., Peptide pulsed dendritic cell vaccination targeting interleukin-13 receptor alpha2 chain in recurrent malignant glioma patients with HLA-A*24/A*02allele. Cytotherapy, 2012. 14(6): p. 733-42.
156. Fong, B., et al., Monitoring of regulatory T cell frequencies and expression of CTLA-4 on T cells, before and after DC vaccination, can predict survival in GBM patients. PLoS One, 2012. 7(4): p. e32614.
157. De Vleeschouwer, S., et al., Stratification according to HGG-IMMUNO RPA model predicts outcome in a large group of patients with relapsed malignant glioma treated by adjuvant postoperative dendritic cell vaccination. Cancer Immunol Immunother, 2012. 61(11): p. 2105-12.
158. Phuphanich, S., et al., Phase I trial of a multi-epitope-pulsed dendritic cell vaccine for patients with newly diagnosed glioblastoma. Cancer Immunol Immunother, 2013. 62(1): p. 125-35.
159. Akiyama, Y., et al., alpha-type-1 polarized dendritic cell-based vaccination in recurrent high-grade glioma: a phase I clinical trial. BMC Cancer, 2012. 12: p. 623.
160. Prins, R. M., et al., Comparison of glioma-associated antigen peptide-loaded versus autologous tumor lysate-loaded dendritic cell vaccination in malignant glioma patients. J Immunother, 2013. 36(2): p. 152-7.
161. Shah, A. H., et al., Dendritic cell vaccine for recurrent high-grade gliomas in pediatric and adult subjects: clinical trial protocol. Neurosurgery, 2013. 73(5): p. 863-7.
162. Reichardt, V. L., et al., Idiotype vaccination using dendritic cells after autologous peripheral blood stem cell transplantation for multiple myeloma—a feasibility study. Blood, 1999. 93(7): p. 2411-9.
163. Lim, S. H. and R. Bailey-Wood, Idiotypic protein-pulsed dendritic cell vaccination in multiple myeloma. Int J Cancer, 1999. 83(2): p. 215-22.
164. Motta, M. R., et al., Generation of dendritic cells from CD14+ monocytes positively selected by immunomagnetic adsorption for multiple myeloma patients enrolled in a clinical trial of anti-idiotype vaccination. Br J Haematol, 2003. 121(2): p. 240-50.
165. Reichardt, V. L., et al., Idiotype vaccination of multiple myeloma patients using monocyte-derived dendritic cells. Haematologica, 2003. 88(10): p. 1139-49.
166. Guardino, A. E., et al., Production of myeloid dendritic cells (DC) pulsed with tumor-specific idiotype protein for vaccination of patients with multiple myeloma. Cytotherapy, 2006. 8(3): p. 277-89.
167. Lacy, M. Q., et al., Idiotype pulsed antigen-presenting cells following autologous transplantation for multiple myeloma may be associated with prolonged survival. Am J Hematol, 2009. 84(12): p. 799-802.
168. Yi, Q., et al., Optimizing dendritic cell-based immunotherapy in multiple myeloma: intranodal injections of idiotype pulsed CD40 ligand-matured vaccines led to induction of type-1 and cytotoxic T-cell immune responses in patients. Br J Haematol, 2010. 150(5): p. 554-64.
169. Rollig, C., et al., Induction of cellular immune responses in patients with stage-I multiple myeloma after vaccination with autologous idiotype-pulsed dendritic cells. J Immunother, 2011. 34(1): p. 100-6.
170. Zahradova, L., et al., Efficacy and safety of Id-protein-loaded dendritic cell vaccine in patients with multiple myeloma—phase II study results. Neoplasma, 2012. 59(4): p. 440-9.
171. Timmerman, J. M., et al., Idiotype-pulsed dendritic cell vaccination for B-cell lymphoma: clinical and immune responses in 35 patients. Blood, 2002. 99(5): p. 1517-26.
172. Maier, T., et al., Vaccination of patients with cutaneous T-cell lymphoma using intranodal injection of autologous tumor-lysate-pulsed dendritic cells. Blood, 2003. 102(7): p. 2338-44.
173. Di Nicola, M., et al., Vaccination with autologous tumor-loaded dendritic cells induces clinical and immunologic responses in indolent B-cell lymphoma patients with relapsed and measurable disease: a pilot study. Blood, 2009. 113(1): p. 18-27.
174. Hus, I., et al., Allogeneic dendritic cells pulsed with tumor lysates or apoptotic bodies as immunotherapy for patients with early-stage B-cell chronic lymphocytic leukemia. Leukemia, 2005. 19(9): p. 1621-7.
175. Li, L., et al., Immunotherapy for patients with acute myeloid leukemia using autologous dendritic cells generated from leukemic blasts. Int J Oncol, 2006. 28(4): p. 855-61.
176. Roddie, H., et al., Phase I/II study of vaccination with dendritic-like leukaemia cells forth immunotherapy of acute myeloid leukaemia. Br J Haematol, 2006. 133(2): p. 152-7.
177. Litzow, M. R., et al., Testing the safety of clinical-grade mature autologous myeloid DC in a phase I clinical immunotherapy trial of CML. Cytotherapy, 2006. 8(3): p. 290-8.
178. Westermann, J., et al., Vaccination with autologous non-irradiated dendritic cells in patients with bcr/abl+ chronic myeloid leukaemia. Br J Haematol, 2007. 137(4): p. 297-306.
179. Hus, I., et al., Vaccination of B-CLL patients with autologous dendritic cells can change the frequency of leukemia antigen-specific CD8+ T cells as well as CD4+CD25+ FoxP3+ regulatory T cells toward an antileukemia response. Leukemia, 2008. 22(5): p. 1007-17.
180. Palma, M., et al., Development of a dendritic cell-based vaccine for chronic lymphocytic leukemia. Cancer Immunol Immunother, 2008. 57(11): p. 1705-10.
181. Van Tendeloo, V. F., et al., Induction of complete and molecular remissions in acute myeloid leukemia by Wilms' tumor 1 antigen-targeted dendritic cell vaccination. Proc Natl Acad Sci USA, 2010. 107(31): p. 13824-9.
182. Iwashita, Y., et al., A phase I study of autologous dendritic cell-based immunotherapy for patients with unresectable primary liver cancer. Cancer Immunol Immunother, 2003. 52(3): p. 155-61.
183. Lee, W. C., et al., Vaccination of advanced hepatocellular carcinoma patients with tumorlysate-pulsed dendritic cells: a clinical trial. J Immunother, 2005. 28(5): p. 496-504.
184. Butterfield, L. H., et al., A phase I/II trial testing immunization of hepatocellular carcinoma patients with dendritic cells pulsed with four alpha-fetoprotein peptides. Clin Cancer Res, 2006. 12(9): p. 2817-25.
185. Palmer, D. H., et al., A phase II study of adoptive immunotherapy using dendritic cells pulsed with tumor lysate in patients with hepatocellular carcinoma. Hepatology, 2009. 49(1): p. 124-32.
186. El Ansary, M., et al., Immunotherapy by autologous dendritic cell vaccine in patients with advanced HCC. J Cancer Res Clin Oncol, 2013. 139(1): p. 39-48.
187. Tada, F., et al., Phase I/II study of immunotherapy using tumor antigen-pulsed dendritic cells in patients with hepatocellular carcinoma. Int J Oncol, 2012. 41(5): p. 1601-9.
188. Ueda, Y., et al., Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. Int J Oncol, 2004. 24(4): p. 909-17.
189. Hirschowitz, E. A., et al., Autologous dendritic cell vaccines for non-small-cell lung cancer. J Clin Oncol, 2004. 22(14): p. 2808-15.
190. Chang, G. C., et al., A pilot clinical trial of vaccination with dendritic cells pulsed with autologous tumor cells derived from malignant pleural effusion in patients with late-stage lung carcinoma. Cancer, 2005. 103(4): p. 763-71.
191. Yannelli, J. R., et al., The large scale generation of dendritic cells for the immunization of patients with non-small cell lung cancer (NSCLC). Lung Cancer, 2005. 47(3): p. 337-50.
192. Ishikawa, A., et al., A phase I study of alpha-galactosylceramide (KRN7000)-pulsed dendritic cells in patients with advanced and recurrent non-small cell lung cancer. Clin Cancer Res, 2005. 11(5): p. 1910-7.
193. Antonia, S. J., et al., Combination of p53 cancer vaccine with chemotherapy in patients with extensive stage small cell lung cancer. Clin Cancer Res, 2006. 12(3 Pt 1): p. 878-87.
194. Perrot, I., et al., Dendritic cells infiltrating human non-small cell lung cancer are blocked at immature stage. J Immunol, 2007. 178(5): p. 2763-9.
195. Hirschowitz, E. A., et al., Immunization of NSCLC patients with antigen pulsed immature autologous dendritic cells. Lung Cancer, 2007. 57(3): p. 365-72.
196. Baratelli, F., et al., Pre-clinical characterization of GMP grade CCL21-gene modified dendritic cells for application in a phase I trial in non-small cell lung cancer. J Transl Med, 2008. 6: p. 38.
197. Hegmans, J. P., et al., Consolidative dendritic cell-based immunotherapy elicits cytotoxicity against malignant mesothelioma. Am J Respir Crit Care Med, 2010. 181(12): p. 1383-90.
198. Um, S. J., et al., Phase I study of autologous dendritic cell tumor vaccine in patients with non-small cell lung cancer. Lung Cancer, 2010. 70(2): p. 188-94.
199. Chiappori, A. A., et al., INGN-225: a dendritic cell-based p53 vaccine (Ad.p53-DC) in small cell lung cancer: observed association between immune response and enhanced chemotherapy effect. Expert Opin Biol Ther, 2010. 10(6): p. 983-91.
200. Perroud, M. W., Jr., et al., Mature autologous dendritic cell vaccines in advanced non-small cell lung cancer: a phase I pilot study. J Exp Clin Cancer Res, 2011. 30: p. 65.
201. Skachkova, O. V., et al., Immunological markers of anti-tumor dendritic cells vaccine efficiency in patients with non-small cell lung cancer. Exp Oncol, 2013. 35(2): p. 109-13.
202. Hernando, J. J., et al., Vaccination with autologous tumour antigen-pulsed dendritic cells in advanced gynaecological malignancies: clinical and immunological evaluation of a phase I trial. Cancer Immunol Immunother, 2002. 51(1): p. 45-52.
203. Rahma, O. E., et al., A gynecologic oncology group phase II trial of two p53 peptide vaccine approaches: subcutaneous injection and intravenous pulsed dendritic cells in high recurrence risk ovarian cancer patients. Cancer Immunol Immunother, 2012. 61(3): p. 373-84.
204. Chu, C. S., et al., Phase I/II randomized trial of dendritic cell vaccination with or without cyclophosphamide for consolidation therapy of advanced ovarian cancer in first or second remission. Cancer Immunol Immunother, 2012. 61(5): p. 629-41.
205. Kandalaft, L. E., et al., A Phase I vaccine trial using dendritic cells pulsed with autologous oxidized lysate for recurrent ovarian cancer. J Transl Med, 2013. 11: p. 149.
206. Lepisto, A. J., et al., A phase I/II study of a MUC1 peptide pulsed autologous dendritic cell vaccine as adjuvant therapy in patients with resected pancreatic and biliary tumors. Cancer Ther, 2008. 6(B): p. 955-964.
207. Rong, Y., et al., A phase I pilot trial of MUC1-peptide-pulsed dendritic cells in the treatment of advanced pancreatic cancer. Clin Exp Med, 2012. 12(3): p. 173-80.
208. Endo, H., et al., Phase I trial of preoperative intratumoral injection of immature dendritic cells and OK-432 for resectable pancreatic cancer patients. J Hepatobiliary Pancreat Sci, 2012. 19(4): p. 465-75.
209. Johnson, T. S. and D. H. Munn, Host indoleamine 2,3-dioxygenase: contribution to systemic acquired tumor tolerance. Immunol Invest, 2012. 41(6-7): p. 765-97.
210. Sternberg, C. N., et al., Progress in the treatment of advanced prostate cancer. Am SocClin Oncol Educ Book, 2014: p. 117-31.
211. Gomella, L. G., F. Gelpi-Hammerschmidt, and C. Kundavram, Practical guide to immunotherapy in castration resistant prostate cancer: the use of sipuleucel-T immunotherapy. Can J Urol, 2014. 21(2 Supp 1): p. 48-56.
212. Tjoa, B. A., et al., Evaluation of phase I/II clinical trials in prostate cancer with dendritic cells and PSMA peptides. Prostate, 1998. 36(1): p. 39-44.
213. Murphy, G. P., et al., Infusion of dendritic cells pulsed with HLA-A2-specific prostate-specific membrane antigen peptides: a phase II prostate cancer vaccine trial involving patients with hormone-refractory metastatic disease. Prostate, 1999. 38(1): p. 73-8.
214. Lodge, P. A., et al., Dendritic cell-based immunotherapy of prostate cancer: immune monitoring of a phase II clinical trial. Cancer Res, 2000. 60(4): p. 829-33.
215. Burch, P. A., et al., Priming tissue-specific cellular immunity in a phase I trial of autologous dendritic cells for prostate cancer. Clin Cancer Res, 2000. 6(6): p. 2175-82.
216. Small, E. J., et al., Immunotherapy of hormone-refractory prostate cancer with antigen-loaded dendritic cells. J Clin Oncol, 2000. 18(23): p. 3894-903.
217. Burch, P. A., et al., Immunotherapy (APC8015, Provenge) targeting prostatic acid phosphatase can induce durable remission of metastatic androgen-independent prostate cancer: a Phase 2 trial. Prostate, 2004. 60(3): p. 197-204.
218. Beinart, G., et al., Antigen-presenting cells 8015 (Provenge) in patients with androgen-dependent, biochemically relapsed prostate cancer. Clin Prostate Cancer, 2005. 4(1): p. 55-60.
219. Kantoff, P. W., et al., Sipuleucel-T immunotherapy for castration-resistant prostate cancer. N Engl J Med, 2010. 363(5): p. 411-22.
220. Barrou, B., et al., Vaccination of prostatectomized prostate cancer patients in biochemical relapse, with autologous dendritic cells pulsed with recombinant human PSA. Cancer Immunol Immunother, 2004. 53(5): p. 453-60.
221. Perambakam, S., et al., Induction of specific T cell immunity in patients with prostate cancer by vaccination with PSA146-154 peptide. Cancer Immunol Immunother, 2006. 55(9): p. 1033-42.
222. Hildenbrand, B., et al., Immunotherapy of patients with hormone-refractory prostate carcinoma pre-treated with interferon-gamma and vaccinated with autologous PSA-peptide loaded dendritic cells—a pilot study. Prostate, 2007. 67(5): p. 500-8.
223. Fuessel, S., et al., Vaccination of hormone-refractory prostate cancer patients with peptide cocktail-loaded dendritic cells: results of a phase I clinical trial. Prostate, 2006. 66(8): p. 811-21.
224. Waeckerle-Men, Y., et al., Dendritic cell-based multi-epitope immunotherapy of hormone-refractory prostate carcinoma. Cancer Immunol Immunother, 2006. 55(12): p. 1524-33.

1. A method of generating a dendritic cell from a stem cell possessing enhanced ability to induce anticancer immunity, wherein said dendritic cell is obtained by the process of: a) selecting a stem cell population; b) genetically modifying said stem cell population to endow enhanced anticancer activity towards said stem cell; c) differentiating said stem cell into a dendritic cell population.

2. The method of claim 1, wherein said stem cell is an inducible pluripotent stem cell.

3. The method of claim 2, wherein said inducible pluripotent stem cell is generated by transfection of mammalian cells with a pluripotency factor, wherein said pluripotency factor gene is one or more genes selected from the group consisting of oct3/4, sox2, klf4, c-myc, lin28, nanog, glis-1, bcl2, bclxl, AIRE, HIF-1 alpha, survivin, livin and bclx.

4. The method of claim 3, wherein, mammalian cells are further provided with: a. a third nucleic acid encoding from 2 to 7 distinct gRNAs, each gRNA comprising a DNA-binding segment and a polypeptide-binding segment, wherein the DNA-binding segment binds the promoter region of a second endogenous pluripotency factor gene; and b. a fourth nucleic acid encoding from 2 to 7 distinct gRNAs, each gRNA comprising a DNA-binding segment and a polypeptide-binding segment, wherein the DNA-binding segment binds the promoter region of a third endogenous pluripotency factor gene; wherein the transcriptional modulator binds the polypeptide-binding segment of the gRNAs encoded by the third and fourth nucleic acids.

5. The method of claim 4, wherein: (i) the DNA-binding segment of each the gRNAs encoded by the first nucleic acid is complementary to at least a portion of the promoter region of a mammalian oct3/4 gene; (ii) the DNA-binding segment of each the gRNAs encoded by the third nucleic acid is complementary to at least a portion of the promoter region of a mammalian sox2 gene; and (iii) the DNA-binding segment of each the gRNAs encoded by the fourth nucleic acid is complementary to at least a portion of the promoter region of a mammalian klf4 gene.

6. The method of claim 1, wherein said pluripotent stem cell is transfected with a tumor antigen in order to induce immunity towards said tumor antigen.

7. The method of claim 6, wherein said tumor antigen is CTCFL.

8. The method of claim 6, wherein said tumor antigen is PDGFR-beta.

9. The method of claim 6, wherein said tumor antigen is PAP.

10. The method of claim 6, wherein said tumor antigen is MAD-CT-2.

11. The method of claim 6, wherein said tumor antigen is Tie-2.

12. The method of claim 6, wherein said tumor antigen is PSA.

13. The method of claim 6, wherein said tumor antigen is protamine.

14. The method of claim 6, wherein said tumor antigen is legumain.

15. The method of claim 6, wherein said tumor antigen is endosialin.

16. The method of claim 6, wherein said tumor antigen is PSMA.

17. The method of claim 6, wherein said tumor antigen is carbonic anhydrase IX.

18. The method of claim 6, wherein said tumor antigen is STn.

19. The method of claim 6, wherein said tumor antigen is Page4.

20. The method of claim 6, wherein said tumor antigen is proteinase 3.
